(19) United States
(12) Patent Application Publication
(10) Pub. No.: US 2025/0255843 A1
(43) Pub. Date: Aug. 14, 2025

(54) COMPOSITIONS COMPRISING CYSTEINE PRODRUGS AND METHODS USING THEREOF

(71) Applicant: Nature's Fusions, LLC, Orem, UT (US)

(72) Inventor: Matthew Peterson, Orem, UT (US)

(21) Appl. No.: 19/038,867

(22) Filed: Jan. 28, 2025

Related U.S. Application Data
(63) Continuation of application No. PCT/US2023/029042, filed on Jul. 28, 2023.
(60) Provisional application No. 63/393,589, filed on Jul. 29, 2022.

(51) Int. Cl. A61K 31/24; A61K 9/00; A61K 9/20; A61K 9/48; A61K 31/197; A61K 33/04; A61K 33/24

(52) U.S. Cl. CPC A61K 31/24; A61K 9/0053; A61K 9/2013; A61K 9/2054; A61K 9/4858; A61K 9/4866; A61K 31/197; A61K 33/04; A61K 33/24

(57) ABSTRACT

Disclosed here are compositions comprising N-acetyl L-cysteine ethyl ester (NACET) and methods of making the same. Food compositions, food supplement, and foodstuff compositions comprising NACET or a capsule comprising NACET are also disclosed.

### CROSS-REFERENCE

This application is a continuation of the International Application No. PCT/US2023/029042, filed Jul. 28, 2023, which claims the benefit of U.S. Provisional Patent Application No. 63/393,589, filed Jul. 29, 2022, all of which are herein incorporated by reference in their entirety for all purposes.

### BACKGROUND

Antioxidants, such as glutathione, may slow the process of oxidation in cells, combat free radicals, and limit disease-causing oxidative stress. Glutathione may work in different organs and/or tissues, including heart, liver, kidney, eye, skin and brain.

Glutathione deficiency can occur and may lead to abnormalities in many biological functions. Despite the advance of nutrition science and medical discoveries, successful prevention or treatment of glutathione deficiency is still needed.

### SUMMARY

Disclosed herein are compositions comprising N-acetyl L-cysteine ethyl ester (NACET) and methods making the same. Once entering the cell and in the presence of certain enzymes, NACET may be transformed into N-acetyl L-cysteine (NAC) by hydrolysis. In turn, NAC may be converted into cysteine, a critical smarting material to make glutathione (GSH). The increase supply of cysteine may lead to the production of GSH in the body.

One aspect of the present disclosure provides a food composition comprising a food carrier and prodrugs of N-acetyl L-cysteine, the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Another aspect of the present disclosure provides a food composition comprising: (a) an effective amount of a prodrug of N-acetyl L-cysteine for modulating levels of cellular glutathione or plasma glutathione or organ glutathione in a subject in need thereof; and (b) a food carrier and/or one or more agents disclosed herein, the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Another aspect of the present disclosure provides a food supplement comprising a prodrug of N-acetyl L-cysteine and a food additive agent, the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Another aspect of the present disclosure provides a method of preparing a food supplement comprising a prodrug of N-acetyl L-cysteine and a food additive agent, comprising mixing the prodrug with said food additive agent, the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

One aspect of the present disclosure provides a foodstuff composition, comprising: (a) a capsule comprising a prodrug of N-acetyl L-cysteine; and (b) a food carrier; the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Another aspect of the present disclosure provides a composition comprising a solid form, a semi-solid form, a capsule, a gummy, or a liquid form, wherein each of said solid form, said semi-solid form, said capsule, said gummy, and said liquid form comprises (a) a prodrug of N-acetyl L-cysteine; and (b) one or more agents; the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Another aspect of the present disclosure provides a composition comprising: (a) a prodrug of N-acetyl L-cysteine; and (b) one or more agents; the prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Disclosed herein are a composition comprising a prodrug of N-acetyl L-cysteine (NAC) including, for example, NACET, a kit, and/or a method that may be useful for purposes described herein, such as maintaining, enhancing, and/or improving health, nutrition, and/or another condition of a subject, such as glutathione deficiency or aging.

In some embodiments, the present disclosure provides a range of physiologically useful concentrations of glutathione to effect a desired physiological effect. In some embodiments, these desired physiological concentrations include serum glutathione concentration at about 2.00 μM, 2.05 μM, 2.10 μM, 2.15 μM, 2.20 μM, 2.25 μM, 2.30 μM, 2.35 μM, 2.40 μM. 2.45 μM, 2.50 μM, 2.65 μM, 2.70 μM, 2.75 μM, 2.80 μM. 2.85 μM, 2.95 μM, 3.00 μM, 3.05 μM, 3.10 μM, 3.15 μM, 3.20 μM, 3.25 μM, 3.30 μM, 3.35 μM, 3.40 μM. 3.45 μM, 3.50 μM, 3.55 μM, 3.60 μM, 3.65 μM, 3.70 μM, 3.75 μM, 3.80 μM. 3.85 μM, 3.95 μM, 4.00 μM, 4.05 μM, 4.10 μM, 4.15 μM, 4.20 μM, 4.25 μM, 4.30 μM, 4.35 μM, 4.40 μM. 4.45 μM, 4.50 μM, 4.55 μM, 4.60 μM, 4.65 μM, 4.70 μM, 4.75 μM, 4.80 μM. 4.85 μM, 4.95 μM, 5.00 μM, 5.05 μM, 5.10 μM, 5.15 μM, 5.20 μM, 5.25 μM, 5.30 μM, 5.35 μM, 5.40 μM. 5.45 μM, 5.50 μM, 5.55 μM, 5.60 μM, 5.65 μM, 5.70 μM, 5.75 μM, 5.80 μM. 5.85 μM, 5.95 μM, 6.00 μM, 6.05 μM, 6.10 μM, 6.15 μM, 6.20 μM, 6.25 μM, 6.30 μM, 6.35 μM, 6.40 μM. 6.45 μM, 6.50 μM, 6.55 μM, 6.60 μM, 6.65 μM, 6.70 μM, 6.75 μM, 6.80 μM. 6.85 μM, 6.95 μM, 7.00 μM, 7.25 μM, 7.5 μM, 7.75 μM, 8.00 μM, 8.25 μM, 8.5 μM, 8.75 μM, 9.00 μM, 9.25 μM, 9.5 μM, 9.75 μM, 10.00 μM, or even higher; plasma glutathione concentration at about 2.00 μM, 2.05 μM, 2.10 μM, 2.15 μM, 2.20 μM, 2.25 μM, 2.30 μM, 2.35 μM, 2.40 μM. 2.45 μM, 2.50 μM, 2.65 μM, 2.70 μM, 2.75 μM, 2.80 μM. 2.85 μM, 2.95 μM, 3.00 μM, 3.05 μM, 3.10 μM, 3.15 μM, 3.20 μM, 3.25 μM, 3.30 μM, 3.35 μM, 3.40 μM. 3.45 μM, 3.50 μM, 3.55 μM, 3.60 μM, 3.65 μM, 3.70 μM, 3.75 μM, 3.80 μM. 3.85 μM, 3.95 μM, 4.00 μM, 4.05 μM, 4.10 μM, 4.15 μM, 4.20 μM, 4.25 μM, 4.30 μM, 4.35 μM, 4.40 μM. 4.45 μM, 4.50 μM, 4.55 μM, 4.60 μM, 4.65 μM, 4.70 μM, 4.75 μM, 4.80 μM. 4.85 μM, 4.95 μM, 5.00 μM, 5.05 μM, 5.10 μM, 5.15 μM, 5.20 μM, 5.25 μM, 5.30 μM, 5.35 μM, 5.40 μM. 5.45 μM, 5.50 μM, 5.55 μM, 5.60 μM, 5.65 μM, 5.70 μM, 5.75 μM, 5.80 μM. 5.85 μM, 5.95 μM, 6.00 μM, 6.05 μM, 6.10 μM, 6.15 μM, 6.20 μM, 6.25 μM, 6.30 μM, 6.35 μM, 6.40 μM. 6.45 μM, 6.50 μM, 6.55 μM, 6.60 μM, 6.65 μM, 6.70 μM, 6.75 μM, 6.80 μM. 6.85 μM, 6.95 μM, 7.00 μM, 7.25 μM, 7.5 μM, 7.75 μM, 8.00 μM, 8.25 μM, 8.5 μM, 8.75 μM, 9.00 μM, 9.25 μM, 9.5 μM, 9.75 μM, 10.00 μM, or even higher or even higher; glutathione concentration in red blood cell (RBC) at about 1,200 μM, 1,300 μM, 1,400 μM, 1,500 μM, 1,600 μM, 1,700 μM, 1,800 μM, 1,900 μM, 2,000 μM, 2,100 μM, 2,200 μM, 2,300 μM, 2,400 μM, 2,500 μM, 2,600 μM, 2,700 μM, 2,800 μM, 2,900 μM, 3,000 μM, 3,100 μM, 3,200 μM, 3,300 μM, 3,400 μM, 3,500 μM, 3,600 μM, 3,700 μM, 3,800 μM, 3,900 μM, 4,000 μM, 4,100 μM, 4,200 μM, 4,300 μM, 4,400 μM, 4,500 μM, 4,600 μM, 4,700 μM, 4,800 μM, 4,900 μM, 5,000 μM, or even higher; and organ glutathione concentration at about 1.0 mM, 1.2 mM, 1.4 mM, 1.6 mM, 1.8 mM, 2.0 mM, 2.2 mM, 2.4 mM, 2.6 mM, 2.8 mM, 3.0 mM, 3.2 mM, 3.4 mM, 3.6 mM, 3.8 mM, 4.0 mM, 4.2 mM, 4.4 mM, 4.6 mM, 4.8 mM, 5.0 mM, 5.2 mM, 5.4 mM, 5.6 mM, 5.8 mM, 6.0 mM, 6.2 mM, 6.4 mM, 6.6 mM, 6.8 mM, 7.0 mM, 7.2 mM, 7.4 mM, 7.6 mM, 7.8 mM, 8.0 mM, 8.2 mM, 8.4 mM, 8.6 mM, 8.8 mM, 9.0 mM, 9.2 mM, 9.4 mM, 9.6 mM, 9.8 mM, 2.0 mM, 9.2 mM, 9.4 mM, 9.6 mM, 9.8 mM, 10.0 mM, or even higher.

In some embodiments, these desired physiological concentrations include serum glutathione concentration at least about 2.00 μM, 2.05 μM, 2.10 μM, 2.15 μM, 2.20 μM, 2.25 μM, 2.30 μM, 2.35 μM, 2.40 μM. 2.45 μM, 2.50 μM, 2.65 μM, 2.70 μM, 2.75 μM, 2.80 μM. 2.85 μM, 2.95 μM, 3.00 μM, 3.05 μM, 3.10 μM, 3.15 μM, 3.20 μM, 3.25 μM, 3.30 μM, 3.35 μM, 3.40 μM. 3.45 μM, 3.50 μM, 3.55 μM, 3.60 μM, 3.65 μM, 3.70 μM, 3.75 μM, 3.80 μM. 3.85 μM, 3.95 μM, 4.00 μM, 4.05 μM, 4.10 μM, 4.15 μM, 4.20 μM, 4.25 μM, 4.30 μM, 4.35 μM, 4.40 μM. 4.45 μM, 4.50 μM, 4.55 μM, 4.60 μM, 4.65 μM, 4.70 μM, 4.75 μM, 4.80 μM. 4.85 μM, 4.95 μM, 5.00 μM, 5.05 μM, 5.10 μM, 5.15 μM, 5.20 μM, 5.25 μM, 5.30 μM, 5.35 μM, 5.40 μM. 5.45 μM, 5.50 μM, 5.55 μM, 5.60 μM, 5.65 μM, 5.70 μM, 5.75 μM, 5.80 μM. 5.85 μM, 5.95 μM, 6.00 μM, 6.05 μM, 6.10 μM, 6.15 μM, 6.20 μM, 6.25 μM, 6.30 μM, 6.35 μM, 6.40 μM. 6.45 μM, 6.50 μM, 6.55 μM, 6.60 μM, 6.65 μM, 6.70 μM, 6.75 μM, 6.80 μM. 6.85 μM, 6.95 μM, 7.00 μM, 7.25 μM, 7.5 μM, 7.75 μM, 8.00 μM, 8.25 μM, 8.5 μM, 8.75 μM, 9.00 μM, 9.25 μM, 9.5 μM, 9.75 μM, or 10.00 μM; plasma glutathione concentration at least about 2.00 μM, 2.05 μM, 2.10 μM, 2.15 μM, 2.20 μM, 2.25 μM, 2.30 μM, 2.35 μM, 2.40 μM. 2.45 μM, 2.50 μM, 2.65 μM, 2.70 μM, 2.75 μM, 2.80 μM. 2.85 μM, 2.95 μM, 3.00 μM, 3.05 μM, 3.10 μM, 3.15 μM, 3.20 μM, 3.25 μM, 3.30 μM, 3.35 μM, 3.40 μM. 3.45 μM, 3.50 μM, 3.55 μM, 3.60 μM, 3.65 μM, 3.70 μM, 3.75 μM, 3.80 μM. 3.85 μM, 3.95 μM, 4.00 μM, 4.05 μM, 4.10 μM, 4.15 μM, 4.20 μM, 4.25 μM, 4.30 μM, 4.35 μM, 4.40 μM. 4.45 μM, 4.50 μM, 4.55 μM, 4.60 μM, 4.65 μM, 4.70 μM, 4.75 μM, 4.80 μM. 4.85 μM, 4.95 μM, 5.00 μM, 5.05 μM, 5.10 μM, 5.15 μM, 5.20 μM, 5.25 μM, 5.30 μM, 5.35 μM, 5.40 μM. 5.45 μM, 5.50 μM, 5.55 μM, 5.60 μM, 5.65 μM, 5.70 μM, 5.75 μM, 5.80 μM. 5.85 μM, 5.95 μM, 6.00 μM, 6.05 μM, 6.10 μM, 6.15 μM, 6.20 μM, 6.25 μM, 6.30 μM, 6.35 μM, 6.40 μM. 6.45 μM, 6.50 μM, 6.55 μM, 6.60 μM, 6.65 μM, 6.70 μM, 6.75 μM, 6.80 μM. 6.85 μM, 6.95 μM, 7.00 μM, 7.25 μM, 7.5 μM, 7.75 μM, 8.00 μM, 8.25 μM, 8.5 μM, 8.75 μM, 9.00 μM, 9.25 μM, 9.5 μM, 9.75 μM, or 10.00 μM; glutathione concentration in red blood cell (RBC) at least about 1,200 μM, 1,300 μM, 1,400 μM, 1,500 μM, 1,600 μM, 1,700 μM, 1,800 μM, 1,900 μM, 2,000 μM, 2,100 μM, 2,200 μM, 2,300 μM, 2,400 μM, 2,500 μM, 2,600 μM, 2,700 μM, 2,800 μM, 2,900 μM, 3,000 μM, 3,100 μM, 3,200 μM, 3,300 μM, 3,400 μM, 3,500 μM, 3,600 μM, 3,700 μM, 3,800 μM, 3,900 μM, 4,000 μM, 4,100 μM, 4,200 μM, 4,300 μM, 4,400 μM, 4,500 μM, 4,600 μM, 4,700 μM, 4,800 μM, 4,900 μM, or 5,000 μM; and organ glutathione concentration at least about 1.0 mM, 1.2 mM, 1.4 mM, 1.6 mM, 1.8 mM, 2.0 mM, 2.2 mM, 2.4 mM, 2.6 mM, 2.8 mM, 3.0 mM, 3.2 mM, 3.4 mM, 3.6 mM, 3.8 mM, 4.0 mM, 4.2 mM, 4.4 mM, 4.6 mM, 4.8 mM, 5.0 mM, 5.2 mM, 5.4 mM, 5.6 mM, 5.8 mM, 6.0 mM, 6.2 mM, 6.4 mM, 6.6 mM, 6.8 mM, 7.0 mM, 7.2 mM, 7.4 mM, 7.6 mM, 7.8 mM, 8.0 mM, 8.2 mM, 8.4 mM, 8.6 mM, 8.8 mM, 9.0 mM, 9.2 mM, 9.4 mM, 9.6 mM, 9.8 mM, 2.0 mM, 9.2 mM, 9.4 mM, 9.6 mM, 9.8 mM, or 10.0 mM.

In some other embodiments, after administer the composition comprising NACET, the physiological glutathione concentration in a subject increases by at least about 10%, 11%, 12%, 13%, 14%, 15%, 20%, 25%, 30%, 35%, 40%, 45%, 50%, 55%, 60%, 65%, 70%, 75%, 80%, 85%, 90%, 95%, 100%, 120%, 140%, 160%, 180%, 200%, 220%, 240%, 260%, 280%, 300%, 320%, 340%, 360%, 380%, 400%, 420%, 440%, 460%, 480%, 500%, or even higher as compared to an initial level of glutathione prior to administration of the composition disclosed herein to the subject. Where desired, suitable concentrations for eliciting the effects of glutathione supplementation as described herein can be from about 0.8, 0.9, 1.0, 1.1, 1.2, 1.3, 1.4, 1.5, 1.6, 1.7, 1.8, 1.9, or 2.0 times the median value reported. Where desired, the selected physiological concentration of glutathione is measured according to the clinically approved methods.

Bioavailability of NACET may be evaluated or measured in any suitable way or using any suitable criterion. Generally, bioavailability of NACET may be evaluated based on NACET absorption rate and/or NACET loading capacity. The NACET absorption rate refers to the fraction of a subject's NACET intake that is absorbed by the subject's body.

Further by way of example, for a given intake of a given composition comprising NACET, there may be an upper limit on the amount of NACET that can be absorbed from the composition comprising NACET by the subject's body within a certain period, such as a 24-hour period. In such a case, as the NACET dosage increases to a certain level, the NACET absorption rate associated with the composition comprising NACET may decline, possibly significantly. Thus, for a given composition comprising NACET, the NACET absorption rate may be suitable when the composition comprising NACET is administered at a relatively low dosage, but may be lower, less suitable, and/or unsuitable at a relatively high dosage.

An upper limit of NACET may be referred to as a NACET loading capacity, which may be used to evaluate the bioavailability of a composition comprising NACET. When a composition that is associated with a relatively low NACET loading capacity is administered to a subject at a relatively high dosage in one case as compared to a relatively low dosage in another case, the NACET absorption rate in the one case may be relatively poorer than a NACET absorption rate in the other case. Thus, for a composition associated with a relatively low NACET loading capacity, a simple increase in dosage may be insufficiently effective or ineffective for efficient NACET intake, provision, and/or supplementation.

A composition comprising NACET with suitably bioavailable may be associated with a suitable or good NACET absorption rate and/or a suitable or good NACET loading capacity. A composition with suitable bioavailability may be provided to a subject in a relatively high dosage in order to provide NACET to a subject with suitable speed. In some embodiments, rapid delivery of NACET may be important in some cases, such as in the treatment of a subject having a severe glutathione deficit and/or another condition amenable to treatment in this manner, for example. Oral administration may be relatively more convenient than intravenous injection in such cases and/or other cases.

The amount of NACET that can be absorbed by a subject, or the rate of absorption of NACET by a subject may vary from subject to subject, based on any of a variety of factors. Examples of such factors include metabolic rate, kidney function, overall health, and/or other factor(s) concerning a subject, and a property or nature of the composition comprising NACET any enhancing agent, its administration vehicle or method, and/or other factor(s) concerning the composition and/or its administration to a subject.

Determining an appropriate dosage for administration of a composition comprising NACET to a subject may take into account any of a variety of factors, such as those just mentioned, for example, any potential or actual side-effect(s), and/or a purpose of the administration of the composition, such as a nutritional or prophylactic purpose, a disease or pathological condition treatment purpose, and/or other purpose(s) for which the composition may be administered to a subject. Determining an appropriate dosage may take into account any of these factors, any other suitable factor(s), any side-effect(s), animal study modeling, human study modeling, clinical study modeling, drug study modeling, and any balancing therebetween.

It is contemplated that a dosage for administration of NACET to a subject may be about 0.075, 0.100, 0.125, 0.150, 0.175, 0.200, 0.225, 0.250, 0.275, 0.300, 0.325, 0.350, 0.375, 0.400, 0.425, 0.450, 0.475, 0.500, 0.525, 0.550, 0.575, 0.600, 0.625, 0.650, 0.675, 0.700, 0.725, 0.750, 0.775, 0.800, 0.825, 0.850, 0.875, 0.900, 0.925, 0.950, 0.975, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.0, 10.5, 11.0, 11.5, 12.0, 12.5, 13.0, 13.5, 14.0, 14.5, or 15.0 mg/kg of body weight/day. For example, it is contemplated that a dosage for administration of NACET to a subject may be from about 5 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100 mg/kg of body weight/day for nutritional and/or prophylactic purpose(s) or for disease and/or pathological condition treatment purpose(s), such as the treatment of glutathione deficiency or aging, for example. Such amounts may be suitable for a human subject, for example.

As mentioned above, such a dosage may be determined, modified and/or refined based on any suitable factor(s), such as results of clinical trials concerning subjects, for example human subjects. In some embodiments, a suitable dosage may be determined, modified and/or refined based on a determination of a suitable dosage for a suitable animal model, based on experimental studies or tests, for example, and conversion of such a suitable animal dosage to a suitable human dosage, based on suitable conversion factor(s), such as any suitable established conversion factor(s), for example. Further by way of example, it is contemplated that any such suitable human dosage may be further determined, modified and/or refined based on clinical trials involving human subjects, for example.

As used herein, a word appearing herein in the singular encompasses its plural counterpart, and a word appearing herein in the plural encompasses its singular counterpart, unless implicitly or explicitly understood or stated otherwise.

As used herein, for any given component described herein, any of the possible candidates or alternatives listed for that component, may generally be used individually or in any combination with one another, unless implicitly or explicitly understood or stated otherwise. Additionally, any list of such candidates or alternatives, is merely illustrative, not limiting, unless implicitly or explicitly understood or stated otherwise.

As used herein, the singular forms “a,” “an,” and “the” include the plural reference unless the context clearly dictates otherwise.

As used herein, the terms “increase” and “decrease” generally refers to, respectively, to cause a statistically significantly (i.e., p<0.1) increase or decrease of at least 5%.

As used herein, when a range of values is provided for a variable, it is to be understood that each intervening value between the upper and lower limit of that range, and any other stated or intervening value in that stated range is encompassed within the scope of the present disclosure. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values≥0 and ≤2 if the variable is inherently continuous.

As used herein, the term “prodrug of N-acetyl L-cysteine” or “prodrug of NAC generally refers to N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

As used herein, the term “capsule” generally refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

As used herein, the term “vegetable capsule” generally refers to a plant capsule shell made from hydroxypropyl methylcellulose (HPMC) extracted from pine or cotton, or other plant sources. Other types of materials that can be used in a vegetable capsule includes hydroxypropyl methylcellulose and carrageenan.

As used herein, the term “tablet” generally refers to a compressed or molded solid dosage form containing the active ingredients with suitable diluents. The tablet can be prepared by compression of mixtures or granulations obtained by wet granulation, dry granulation or by compaction well known to a person skilled in the art.

As used herein, the term “oral gel” generally refers to the active ingredients dispersed or solubilized in a hydrophilic semi-solid matrix.

As used herein, unless specifically indicated otherwise, the word “or” is used in the inclusive sense of “and/or” and not the exclusive sense of “either/or”.

As used herein, the term “treatment” generally refers to the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

As used herein, the terms “approximately”, “about” and the symbol “˜” in reference to a figure or number or amount include numbers that fall within a range of ±10% of same, unless implicitly or explicitly understood or stated otherwise.

As used herein, it is to be understood that any permissive, open, or open-ended language encompasses any relatively permissive to restrictive language, less open to closed language, or less open-ended to closed-ended language, respectively, unless implicitly or explicitly understood or stated otherwise. Merely by way of example, the word “comprising” may encompass “comprising”-, “consisting essentially of”-, and/or “consisting of”-type language.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment.

### Glutathione and N-Acetyl Cysteine

Glutathione (GSH) is found in almost every cell in the body, and it is a central component of the oxidative-reductive (redox) machinery of every cell. One of its key functions is to combine with, and thereby inactivate (or detoxify) certain chemical agents in the body. These chemical agents may include reactive oxidative intermediates (ROI), carcinogens, oxidative molecules, some drugs, exogenous chemicals and toxins. GSH or the lack thereof can be associated with inflammation, free radical protection, oxidative stress, aging, retinal pigment production. GSH supports cell health for brain, heart, lungs, all other organs and tissues.

GSH depletion may impact a wide variety of cellular processes, including, but not limited to, DNA synthesis, gene expression, sugar metabolism, and lactate production. In mammals, GSH also participates in regulating the expression or activity of extracellular molecules, such as cytokines and adhesion molecules implicated in inflammatory reactions and other disease processes. Because GSH is depleted in these detoxification reactions, it must be continually replenished to maintain at a healthy level to support normal cellular functions. Acute GSH depletion may cause severe, sometimes fatal, oxidative and/or alkylation injury.

Production of GSH decreases as people grow older. For example, GSH content in lymphocytes are found to decline with age. From the ages of 20-40 to 60-80, the decrease of GSH is 36% in females and 42% in males. The enzyme activity of glutathione S-transferase (GST) was found to decrease up to 58% as people age. Reasons for the age-related decease in GSH include increased rate of oxidation in senescence, decreased GSH synthesis due to cysteine deficiency, and/or diminished activity of glutamyl-cysteine synthetase and increased GSH consumption in the removal of peroxides and xenobiotics.

Taking GSH as a supplement does not help because GSH has poor bioavailability. One possible reason for the poor oral bioavailability for GSH may include the action of an intestinal enzyme, the γ-glutamyl transpeptidase (GGT) which degrades GSH.

GSH is a tripeptide-glutamyl-cysteinyl-glycine, made from cysteine, glycine, and glutamic acid. Biosynthesis of GSH requires cysteine, which is a conditionally essential amino acid that must be obtained from dietary sources or by conversion of dietary methionine via the cystathionase pathway. Normal GSH levels can be maintained when the supply of cysteine from nutrients is adequate. However, if the supply of cysteine is inadequate to maintain GSH homeostasis and the consumption of GSH increases, GSH depletion occurs. The physiological concentration of GSH is in the millimolar level in most cells. Glutathione exists in cells in 2 states: reduced (GSH) and oxidized (GSSG). Oxidized glutathione is actually 2 reduced glutathiones bound together at the sulfur atoms (forming a disulfide bond).

Orally taking cysteine does not help, either. Oral cysteine is limited by spontaneous oxidation of cysteine to its corresponding disulfide cysteine, which may cause significant toxicity, and increase mutagenicity risks.

N-acetylcysteine (NAC) is an efficient non-toxic source of cysteine. Treatment with NAC can replenish hepatocellular GSH in certain case of acute GSH depletion such as acetaminophen poisoning. In addition, treatment with NAC provides beneficial effects in respiratory, cardiovascular, endocrine and infectious and other disease settings. For example, rapid administration of NAC is the standard of care for preventing hepatic injury in acetaminophen overdose. NAC is about 10 times more stable than cysteine and much more soluble than the stable cysteine disulfide, cystine. NAC has been approved for therapeutic use for treatment of acetaminophen overdose and as a mucolytic agent in cystic fibrosis. NAC is mainly considered to be an antioxidant rather than a source of cysteine for GSH replenishment. While antioxidants, such as vitamin E and vitamin C, can spare GSH under conditions of oxidative stress, GSH loss due to oxidative or detoxifying reactions can only be offset by GSH resynthesis, which requires a cysteine source such as NAC. NAC can be administered by intravenous, enteral, and rectal routes.

However, NAC has a bioavailability of only 4-6%. Orally, NAC is readily absorbed by the stomach and gut, and then converted to cysteine in the liver, where it is majorly used up, with small amounts transported to other tissues. Pharmacokinetics studies have revealed that NAC is metabolized in liver and kidney. Hence, NAC is almost undetectable in plasma, which is where it would have done the most benefit. NAC is poorly membrane penetrable so even when it makes it to the cell it cannot increase the GSH levels except at extremely high dosages. Nor can NAC to cross blood-brain barrier.

### N-Acetylcysteine Ethyl Ester

N-Acetyl-L-cysteine ethyl ester (NACET) is a prodrug form of NAC and an esterified analog of NAC. Studies have shown that medium to high doses (up to 8 g/day) of NAC may result in 5-20% increase of plasma. In contrast, NACET at lower than 8 g/day doses may increase plasma GSH by 250%. NACET has a bioavailability of 60-80% or more, about 10-20 times that of NAC (4-6%).

Masking the carboxylic acid by an ester causes the NACET to be recognized by the body as something other than NAC. Consequently, the liver and kidneys do not metabolize NACET in the same way they metabolize NAC. NACET can be absorbed by the red blood cells and to be transported though the entire body including crossing the blood-brain barrier. Thus, NACET, but not NAC, can help protect the brain and eyes from free radicals and help with cell regeneration. As a lipophilic cell-permeable cysteine derivative, NACET acts as a protective agent against oxidative damage at a 5-10 times lower concentration than that of NAC. Accordingly, NACET is a component of compositions disclosed herein.

The amount of NACET in a capsule may vary. The amount of NACET in a capsule may be about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg.

The amount of NACET in a capsule may be from about 1 mg to about 2 mg, from about 2 mg to about 5 mg, from about 5 mg to about 10 mg, from 10 mg to about 20 mg, from about 20 mg to about 30 mg, from about 30 mg to about 40 mg, from about 40 mg to about 50 mg, from about 50 mg to about 60 mg, from about 60 mg to about 70 mg, from about 70 mg to about 80 mg, from about 80 mg to about 90 mg, from about 90 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 550 mg to about 550 mg, from about 550 mg to about 600 mg, from about 600 mg to about 650 mg, about from about 650 mg to 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1,000 mg.

Other prodrugs of NAC may be used to increase or modulate glutathione levels as what is described herein for NACET. The concentrations or formulations of each prodrug of NAC may be the same as or different from those of NACET, may be independently determined, tested, and/or optimized for each prodrug.

### Glycine

Biosynthesis of GSH requires glycine. In addition, glycine may be used to create proteins for tissue maintenance, hormone creation, and enzymes synthesis. Accordingly, glycine can be co-administered together with NACET.

The amount of glycine co-administered with NACET and/or the amount of glycine in a capsule together with NACET may vary. The amount of glycine co-administered with NACET and/or in a capsule together with NACET may be about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg.

The amount of glycine co-administered with NACET and/or administered in a capsule together with NACET may be from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 550 mg to about 550 mg, from about 550 mg to about 600 mg, from about 600 mg to about 650 mg, about from about 650 mg to 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1,000 mg.

Other prodrugs of NAC may be used together with glycine to increase or modulate glutathione levels as what is described herein for NACET.

### Selenium

Selenium is an essential trace mineral that may enhance the functions of white blood cells and t-cells. It may improve performance of the overall immune system of the body. Further, selenium is part of the glutathione peroxidase and other selenoenzymes or selenoproteins, which are involved in the removal of hydrogen peroxide and lipid peroxide produced during oxidative stress. Some studies of age-related differences in blood selenium levels have reported that the aging process is a significant factor in the decrease of serum selenium levels. For example, selenium supports thyroid health, cognitive function, immune health, and free radical protection. Selenium can be administered together with NACET.

The amount of selenium co-administered with NACET and/or the amount of selenium in a capsule together with NACET may vary. The amount of selenium co-administered with NACET and/or in a capsule together with NACET may be about 1 μg, about 2 μg, about 3 μg, about 4 μg, about 5 μg, about 6 μg, about 7 μg, about 8 μg, about 9 μg, about 10 μg, about 12 μg, about 14 μg, about 16 μg, about 18 μg, about 20 μg, about 25 μg, about 30 μg, about 35 μg, about 40 μg, about 45 μg, about 50 μg, about 55 μg, about 60 μg, about 65 μg, about 70 μg, about 75 μg, or about 80 μg.

The amount of selenium co-administered with NACET and/or administered in a capsule together with NACET may be from about 1 μg to about 2 μg, from about 2 μg to about 3 μg, from about 3 μg to about 4 μg, from about 4 μg to about 5 μg, from about 5 μg to about 6 μg, from about 6 μg to about 7 μg, from about 7 μg to about 8 μg, from about 8 μg to about 9 μg, from about 9 μg to about 10 μg, from about 10 μg to about 12 μg, from about 12 μg to about 14 μg, from about 14 μg to about 16 μg, from about 16 μg to about 18 μg, from about 18 μg to about 20 μg, from about 20 μg to about 25 μg, from about 25 μg to about 30 μg, from about 30 μg to about 35 μg, from about 35 μg to about 40 μg, from about 40 μg to about 45 μg, from about 45 μg to about 50 μg, from about 50 μg to about 55 μg, from about 55 μg to about 60 μg, from about 60 μg to about 65 μg, from about 65 μg to about 70 μg, from about 70 μg to about 75 μg, or from about 75 μg to about 80 μg.

Other prodrugs of NAC may be used together with selenium to increase or modulate glutathione levels as what is described herein for NACET.

### Molybdenum

Molybdenum works in the body to break down proteins such that certain enzymes then use to break down toxins in the body. Molybdenum helps remove bodily toxins. It is an essential trace mineral that is not naturally produced by the body. Molybdenum can be administered together with NACET.

The amount of molybdenum co-administered with NACET and/or the amount of molybdenum in a capsule together with NACET may vary. The amount of molybdenum co-administered with NACET and/or in a capsule together with NACET may be about 1 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg.

The amount of molybdenum co-administered with NACET and/or administered in a capsule together with NACET may be from about 1 μg to about 5 μg, from about 5 μg to about 10 μg, from about 10 μg to about 15 μg, from about 15 μg to about 20 μg, from about 20 μg to about 25 μg, from about 25 μg to about 30 μg, from about 30 μg to about 35 μg, from about 35 μg to about 40 μg, from about 40 μg to about 45 μg, from about 45 μg to about 50 μg, from about 50 μg to about 55 μg, from about 55 μg to about 60 μg, from about 60 μg to about 65 μg, from about 65 μg to about 70 μg, from about 70 μg to about 75 μg, from about 75 μg to about 80 μg, from about 80 μg to about 85 μg, from about 85 μg to about 90 μg, from about 90 μg to about 95 μg, or from about 95 μg to about 100 μg.

Other prodrugs of NAC may be used together with molybdenum to increase or modulate glutathione levels as what is described herein for NACET.

### Essential Oils

As used herein, the term “essential oil” refers to botanical oils and lipids. Non-limiting examples of essential oils are anise oil, angelica oil, basil oil, bay oil, bay laurel oil, bergamot oil, bois de rose oil, California bay laurel oil, camphor oil, cananga oil, cannabis oil, cardamom oil, caraway oil, cedar oil, cedarwood oil, Chamaecyparis obtusa oil, chamomile oil, cinnamon oil, citronella oil, clove oil, copaiba balsam oil, cumin oil, clove oil, coriander oil, dill oil, eucalyptus oil, fennel oil, garlic oil, geranium oil, grapefruit oil, ginger oil, glycerol-derived lipids or glycerol fatty acid derivatives, guaiac oil, hiba oil, Indonesian bay leaf oil, iris oil, Japanese mint oil, jasmine oil, lavender oil, lemon oil, lemongrass oil, linaloe oil, Lindera oil, marjoram oil, mandarin oil, melaleuca oil, Mexican bay leaf oil, mint oil, neroli oil, onion oil, orange oil, oregano oil, palmarosa sofia oil, patchouli oil, parsley oil, pepper oil, peppermint oil, perilla oil, Peru balsam oil, petitgrain oil, pine oil, pine needle oil, pyrethrum, rose oil, rosemary oil, sage oil, sandalwood oil, sesame oil, spearmint oil, star anis oil, sweet orange oil, tangerine oil, tea seed oil, tea tree oil, thyme oil, tolu balsam oil, tuberose oil, turmeric oil, vetivert oil, West Indian bay tree oil, western mint oil, or wintergreen oil and others as disclosed herein throughout.

Other prodrugs of NAC may be used together with the essential oil to increase or modulate glutathione levels as what is described herein for NACET.

### Diseases and Conditions

As used herein, diseases and conditions associated with glutathione deficiency may include, but are not limited to, chronic obstructive pulmonary disease (COPD), acute renal failure, sickle cell anemia, diabetes mellitus, inflammatory diseases, human immunodeficiency virus mediated disease, malaria protein-energy malnutrition, otic disease, neurodegenerative disease, and cardiovascular disease.

### Additional Therapeutic Agent

As used herein, additional therapeutic agents may include, but not limited to, a therapeutic agent to treat and/or prevent chronic obstructive pulmonary disease (COPD), acute renal failure, sickle cell anemia, diabetes mellitus, inflammatory diseases, human immunodeficiency virus mediated disease, malaria protein-energy malnutrition, otic disease, neurodegenerative disease, and cardiovascular disease. Any of these additional therapeutic agents may be co-administered with NACET either simultaneously or sequentially.

Other prodrugs of NAC may be co-administered together with the additional therapeutic agent to increase or modulate glutathione levels as what is described herein for NACET.

### Compositions

The following compositions recite NACET as an example. Other prodrugs of NAC may be used in compositions to increase or modulate glutathione levels as what is described herein for NACET.

It is contemplated that a composition comprising NACET described herein may be administered to a human subject to suitable or beneficial effect, such as nutritional, prophylactic, and/or therapeutic effect, in any suitable manner. In some embodiments, a composition comprising NACET of the present disclosure may be administered to a human subject susceptible to, or afflicted by, glutathione deficiency to suitable or beneficial effect. In other embodiments a composition comprising NACET, or a composition containing such a compound, may be administered to a human subject for a variety of useful purposes, such as anti-aging. As the composition comprising NACET comprises a derivative of an amino acid, and may comprise other natural ingredients, such as an enhancing agent described herein, for example, in most embodiments administration of the composition comprising NACETs of the present disclosure may be safe over a relatively long term. In still other embodiments, administration of such a composition comprising NACET occurs over a long-term period. For example, a subject may be administered the compositions of the present disclosure for weeks, months, years, and/or for life. Such long-term administration may be used for preventing or treating a condition, such as glutathione deficiency, or may be useful for preventing progression of a condition (e.g., preventing the progression of aging). These examples are not limiting examples, as long-term administration of the composition comprising NACETs of the present disclosure may be used for multiple purposes as described herein and as recognized by one of skill in the art.

A composition comprising NACET described herein may comprise one or more other suitable component(s), such as a suitable pharmaceutical composition or drug associated with the treatment of glutathione deficiency, including, for example, acute renal failure, Alzheimer's disease, cancer, cardiovascular disease, chronic obstructive pulmonary disease (COPD), diabetes mellitus, inflammatory diseases, diseases of aging, human immunodeficiency virus mediated disease, intellectual disability, loss of coordination, malaria protein-energy malnutrition, metabolic disease, obesity, otic disease, neurodegenerative disease, oxidative stress disease, Parkinson's disease, seizure, and sickle cell anemia. A composition comprising NACET of the present disclosure may be effective in the treatment of certain diseases. A subject afflicted with the disease may have a glutathione deficiency, which may be addressed by another therapeutic agent to treat the affliction. It is contemplated that the other therapeutic agent and a composition comprising NACET described herein may work synergistically in a suitable manner, such as a biologically beneficial and/or a therapeutically effective manner.

In some embodiments, the composition comprising NACET described herein can be used to treat or alleviate the symptoms of RBC-GSH deficiency, elevated oxidative stress (OxS), mitochondrial dysfunction such as impaired mitochondrial fuel-oxidation (MFO), obsessive compulsive disorder (OCD) related skin picking, anxiety, chronic fatigue, and autoimmune condition with systemic inflammation; improve inflammation, endothelial dysfunction, insulin-resistance, recovery from toxic exposure, bloating, reduced mood swing, recovery from cytokine storm from COVID, recovery from cold or flu, recovery from sinus drippage, genomic-damage, cognition, memory, strength, gait-speed, and exercise capacity; and lower body-fat and waist-circumference.

A composition comprising NACET appropriate for administration to a subject may be administered in any suitable manner. Such administration may be oral and/or any other suitable administration, such as transdermal, intramuscular, vaginal, rectal, subdermal. Components of a composition comprising NACET, such as at least one composition comprising NACET may be administered to a subject concurrently, such as in any manner of concurrent administration described herein and/or in U.S. Patent Application Publication No. 2006/0089335 A1, the content of which is incorporated by reference in its entirety.

A composition comprising NACET appropriate for administration to a subject may be provided in any suitable form, such as a liquid form, a gel form, a semi-liquid (for example, a liquid, such as a viscous liquid, containing some solid) form, a semi-solid (a solid containing some liquid) form, and/or a solid form, for example. Merely by way of example, a tablet form, a capsule form, an oral gel form, a food form, a chewable form, a non-chewable form, a slow- or sustained-release form, a non-slow- or non-sustained-release from, and/or the like, may be employed. Gradual-release tablets are known in the art. Examples of such tablets are set forth in U.S. Pat. No. 3,456,049, the content of which is incorporated by reference in its entirety. Such a composition may comprise an additional agent or agents, whether active or passive. Examples of such an agent include a sweetening agent, a flavoring agent, a coloring agent, a filling agent, a binding agent, a lubricating agent, an excipient, a preservative, a manufacturing agent, and/or the like, merely by way of example, in any suitable form. A slow- or sustained-release form may delay disintegration and/or absorption of the composition and/or one or more component(s) thereof over a period, such as a relatively long period, for example. A food form may take the form of a food bar, a cereal product, a bakery product, a dairy product, and/or the like, for example. A bakery product form may take the form of a bread-type product, such as a bagel or bread itself, for example, a donut, a muffin, and/or the like, merely by way of example. A component of a composition comprising NACET may be provided in a form that is other than that of another component of the composition comprising NACET. For example, at least one composition comprising NACET may be provided in a solid form, such as solid food or cereal that is taken with an enhancing agent in a liquid form, such as a liquid dietary substance. Such administration of composition comprising NACETs in multiple forms, may occur simultaneously (e.g., ingesting a NACET tablet with NACET-fortified milk), or at different times.

In some embodiments, a composition comprising NACET in the form of a pill, tablet, capsule, or like device, may comprise from about 1 mg to about 1,000 mg of NACET. In other embodiments, a composition comprising NACET may contain from about 1 mg to about 100 mg of NACET. In still other embodiments, a composition comprising NACET in the form of a food serving, or like dietary serving, may comprise from about 20 mg to about 80 mg of NACET. In still other embodiments, a composition comprising NACET in the form of a food serving, or like dietary serving, may comprise from about 45 mg to about 55 mg of NACET.

A composition comprising NACET appropriate for administration to a subject may be provided in a liquid form, such as one suitable for oral administration, parenteral administration and/or other appropriate routes. Such a composition may comprise any suitable additional agent or agents, whether active or passive. Examples of such agents include water, a sweetening agent, a flavoring agent, a coloring agent, a texturing agent, a stabilizing agent, a preservative, a manufacturing agent, and/or the like, in any suitable form. A composition comprising NACET in a liquid form may comprise from about 5 mg/L to about 100 g/L, such as from about 5 mg/L to about 50 mg/L, from about 50 mg/L to about 12 g/L, from about 12 g/L to about 20 g/L, from about 20 g/L to about 40 g/L, from about 40 g/L to about 60 g/L, from about 60 g/L to about 80 g/L, from about 80 g/L to about 100 g/L, for example, of NACET. An amount of from about 50 mg/L to about 3 g/L, such as from about 100 mg/L to about 1.5 g/L, for example, of NACET may be suitable for prophylactic application and/or nutritional application. An amount of from about 300 mg/L to about 12 g/L, such as from about 500 mg/L to about 3.5 g/L, for example, of NACET may be suitable for therapeutic application.

A composition comprising NACET in a liquid form may be used in any suitable manner. In some embodiments, the composition comprising NACET may be used as a beverage, such as a milk-based beverage, a sports drink, a fruit juice drink, an alcoholic beverage, and/or the like. In other embodiments, the composition comprising NACET in liquid form contains multiple composition comprising NACETs. In such embodiments, the weight percentage of each composition comprising NACET may vary in relation to the other. In still other embodiments, the composition comprising NACET in a liquid form may take the form of a magnesium-fortified product comprising water, NACET, and optionally, at least one agent sufficient to confer a suitable property to the product. In still another embodiment, a composition comprising NACET in a liquid form may be formulated from a dry mix, such as a dry beverage mix or a NACET-fortified, milk-comprising powder. A dry mix may be suitable in terms of transportation, storage, and/or shelf life. The composition may be formulated from the dry mix in any suitable manner, such as by adding a suitable liquid (e.g., water, milk, fruit juice, alcohol, etc.).

Examples concerning composition comprising NACET(s) and composition comprising NACET(s), and the preparation, testing and/or use of same, are provided below.

### Use as Dietary Supplement

One embodiment of the present disclosure is a NACET dietary supplement. In some embodiments, the NACET supplement contains one or more composition comprising NACETs of the present disclosure and may optionally contain other ingredients generally recognized as safe for food additive use, including, but not limited to, preservatives (e.g., butylated hydroxytoluene, butylated hydroxyanisole), food grade emulsifiers (e.g., lecithin, propylene glycol esters), and pharmaceutically acceptable carriers and excipients (e.g., binders, fillers, lubricants, dissolution aids).

In one embodiment, the NACET supplement composition of the present disclosure is made by combining NACET of the disclosure, as well as any optional components, in the desired relative amounts and mixing the components according to known methods to produce a substantially homogeneous mixture.

In another embodiment, the composition comprising NACET may also contain other nutritional active materials including, without limitation, calcium-containing materials such as calcium carbonate, stannol esters, hydroxycitric acid, vitamins, minerals, herbals, spices and mixtures thereof. Examples of vitamins that are available as additional ingredients include, but are not limited to, vitamin A (retinol), vitamin D (cholecalciferol), vitamin E group (alpha-tocopherol and other tocopherols), vitamin K group (phylloquinones and menaquinones), thiamine (vitamin B1), riboflavin (vitamin B2), niacin, vitamin B6 group, folic acid, vitamin B12 (cobalamins), biotin, vitamin C (ascorbic acid), and mixtures thereof. The amount of vitamin or vitamins present in the final product is dependent on the particular vitamin. Examples of minerals that are available as additional ingredients include, but are not limited to, calcium, magnesium, phosphorus, iron, zinc, iodine, selenium, potassium, copper, manganese, molybdenum and mixtures thereof. As is the case with vitamins, the amount of mineral or minerals present in the final product is dependent on the particular mineral. The present list of additional neutriceutical components are provided by way of example only and are not intended to be limiting.

In yet another embodiment, the present dietary supplement or food compositions are formulated to have suitable and desirable taste, texture, and viscosity for consumption. Any suitable food carrier can be used in the present food compositions. Food carriers of the present disclosure include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables.

In an embodiment, food carriers employed herein can mask the undesirable taste (e.g., bitterness), if present in one or more of the subject composition comprising NACETs. Where desired, the food composition presented herein exhibit more desirable textures and aromas than that of the composition comprising NACETs.

For example, liquid food carriers may be used according to the disclosure to obtain the present food compositions in the form of beverages, such as supplemented juices, coffees, teas, and the like. In other embodiments, solid food carriers may be used according to the disclosure to obtain the present food compositions in the form of meal replacements, such as supplemented snack bars, pasta, breads, and the like. In yet other embodiments, semi-solid food carriers may be used according to the disclosure to obtain the present food compositions in the form of gums, chewy candies or snacks, and the like.

In another embodiment, the supplement composition of the present disclosure may be administered in any oral dosage form, including liquid dosage forms (e.g., a suspension or slurry), and oral solid dosage forms (e.g., a tablet or bulk powder). As used herein the term “tablet” refers generally to tablets, caplets, capsules, including soft gelatin capsules, and lozenges.

Tablets are made by methods known in the art and may further comprise suitable binders, lubricants, diluents, disintegrating agents, colorants, flavoring agents, flow-inducing agents, melting agents which are known in the art. The oral solid dosage form may, optionally, have a film coating to protect the components of the NACET composition from one or more of moisture, oxygen and light or to mask any undesirable taste or appearance. Suitable coating agents include, for example, cellulose, hydroxypropylmethyl cellulose. Where desired, tablets can be formulated in sustained release format. Methods of making sustained release tablets are known in the art, e.g., see US2006051416 and US20070065512, both of which are incorporated herein by reference.

In still other embodiments, composition comprising NACETs of the present disclosure are added to foodstuffs. Such foodstuffs may be naturally high or low in cysteine.

### Use as Pharmaceutical

One embodiment of the present disclosure is a pharmaceutical composition, typically for administration to a subject in need of therapeutic levels of glutathione. Various delivery systems can be used to administer the NACET compositions of the disclosure, e.g., encapsulation in liposomes, microparticles, microcapsules, etc. Methods of delivery include but are not limited to intra-arterial, intramuscular, intravenous, intranasal, and oral routes. In a specific embodiment, it may be desirable to administer the pharmaceutical compositions of the disclosure locally to the area in need of treatment; this may be achieved by, for example, and not by way of limitation, transdermal patches, local infusion during surgery, by injection, or by means of a catheter (with or without an attached pump).

In some embodiments, administration of the composition comprising NACET can be effected in one dose, continuously or intermittently throughout the course of treatment. Methods of determining the most effective means and dosage of administration may vary with the composition used for therapy, the purpose of the therapy, the target cell or tissue being treated, and the subject being treated. Single or multiple administrations can be carried out with the dose level and pattern being selected by the treating physician.

For oral administration, the compositions may optionally be formulated by mixing the compositions with physiologically or pharmaceutically acceptable carriers. Such oral dosage forms may be formulated as tablets, pills, dragees, capsules, emulsions, lipophilic and hydrophilic suspensions, liquids, gels, syrups, slurries, suspensions and the like, for oral ingestion by an individual or a patient to be treated.

In one embodiment, the composition is contained in capsules. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as tale or magnesium stearate and, optionally, stabilizers. Optionally, the inventive composition for oral use can be obtained by mixing the composition with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For buccal administration, the inventive compositions may take the form of tablets or lozenges formulated in a conventional manner. For administration by inhalation, the compositions of the present disclosure may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The preparation of pharmaceutical compositions of this disclosure is conducted in accordance with generally accepted procedures for the preparation of pharmaceutical preparations. See, for example, Remington's Pharmaceutical Sciences 18th Edition (1990), E. W. Martin ed., Mack Publishing Co., PA, the content of which is incorporated by reference in its entirety. Depending on the intended use and mode of administration, it may be desirable to process the composition comprising NACET further in the preparation of pharmaceutical compositions. Appropriate processing may include mixing with appropriate non-toxic and non-interfering components, sterilizing, dividing into dose units, and enclosing in a delivery device.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

In some embodiments, NACET supplementation is provided to achieve optimal body glutathione status by supplementing a person's diet with a NACET composition of the present disclosure. As described herein, there is a desired range of physiological glutathione level, below which and above which, detrimental effects may occur. In some embodiments, use of the compositions disclosed herein may allow a subject to maintain a dosage regimen which allows for a physiological concentration as high as possible, without encountering detrimental effects. Desired body glutathione status may be applicable for general health as well as for specific therapeutic applications described herein. It is to be understood that for treatment of different conditions, the optimal body glutathione status may be different to achieve the desired effects. The compositions described herein can be utilized for the methods described herein to achieve therapeutically effective glutathione concentrations.

The pharmaceutical compositions can be formulated in slow release or sustained release forms, whereby a relatively consistent level of the active compound is provided over an extended period. In some embodiments, a composition comprising NACET and other therapeutic agents may be administered jointly or separately by using a controlled release dosage form. Controlled release within the scope of this disclosure can be taken to mean any one of a number of extended-release dosage forms. Extended-release dosage forms are described in Heaton et al., U.S. Patent Application Pub. No. 2005/0129762 A1 and Edgren et al. U.S. Patent Application Pub. No. 2007/0128279 A1, each of which is herein incorporated by reference in its entirety. Time-release formulations are known in the art and are described in Sawada et al. U.S. Patent Application Pub. No. 2006/0292221 A1, which is herein incorporated by reference in its entirety. The following terms may be considered to be substantially equivalent to controlled release for the purposes of the present disclosure: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.). The various controlled release technologies cover a very broad spectrum of drug dosage forms. Controlled release technologies include, but are not limited to, physical systems and chemical systems.

### Excipient

Various excipients can be used to prepare the compositions comprising NACET. Direct compression tablet manufacturing may be used to prepare compositions comprising NACET using an excipient. It is a simple process involving less extensive equipment, operating time and cost. Microcrystalline cellulose is one example of an excipient for direct compression processing. Microcrystalline cellulose has inherently high compressibility due to its plastic deformation and limited elastic recovery. Microcrystalline cellulose usually provides for good drug dispersion, even ordered mixing with some drugs and particular grades of microcrystalline cellulose. However, the material flow properties are relatively poor for most grades of microcrystalline cellulose. Intermittent and non-uniform flow can occur as the formulation moves from the hopper to the die on a tablet press. This non-uniform flow can lead to drug content variations in the finished tableted dosage form.

In some embodiments, a wet granulation process is utilized. The popularity of the wet granulation process as compared to the direct compression process is based on at least three potential advantages. First, wet granulation may provide the material to be compacted with a more hydrophilic nature, in order to improve the wetting, disintegration and dissolution characteristics of some hydrophobic drugs or ingredients. Second, the content uniformity and drug segregation-resistance can be enhanced using a granulation step to lock drug and excipient components together during blending. Finally, the micrometric characteristics of the component powders can be optimized prior to compaction, which is often aided by incorporation of a polymeric binder. It is normally considered that this last property imbued by wet granulation yields a significantly more compactable product and consequently stronger, more robust tablets.

Depending upon the amount and type of drying, the concentration of the NACET in the form of a wet cake and any augmenting agents present, the compressible particles may have different particle sizes, densities, pH, moisture content, etc. Possible excipients used in the current disclosure may include, but not be limited to, beta-cyclodextrin, calcium phosphate (dibasic), calcium silicate, calcium stearate, carboxymethyl cellulose (carmellose), carmellose sodium, croscarmellose, ethyl cellulose, dextrin, D-mannitol, D-sorbitol, erythritol, ethyl cellulose, hydroxy propyl methylcellulose, kaolin, lactose, maltose, magnesium silicate, magnesium aluminometasilicate, microcrystalline cellulose, powdered hydrogenated maltose starch syrup, maltitol, powdered sucrose, povidone, pregelatinized starch, shellac (and glaze), silicic acid or a salt thereof, silicon dioxide, sodium carboxymethyl cellulose (carmellose sodium), starch (corn), sodium starch clycolate, stearic acid, sucrose, talc, titanium dioxide, wheat starch, white soft sugar, or xylitol, or a combination thereof. The excipient content is not particularly limited and may be from about 10 to about 98.5% by weight with respect to the weight of the tablet (when the tablet is coated, the weight of the uncoated tablet), preferably from about 20 to about 95%, and more preferably from about 30 to about 90% by weight.

Surfactants which may be used in the present disclosure as a compressibility augmenting agent generally include all pharmaceutically-acceptable surfactants. Suitable pharmaceutically-acceptable anionic surfactants include, for example, those containing carboxylate, sulfonate, and sulfate ions. Those containing carboxylate ions are sometimes referred to as soaps and are generally prepared by saponification of natural fatty acid glycerides in alkaline solutions. The most common cations associated with these surfactants are sodium, potassium, ammonium and triethanolamine. The chain length of the fatty acids ranges from 12 to 18. Although a large number of alkyl sulfates are available as surfactants, one particularly preferred surfactant is sodium lauryl sulfate, which has an HLB value of about 40.

In the pharmaceutical arts, sodium lauryl sulfate has been used as an emulsifying agent in amounts of up to about 0.1% by weight of the formulation. Sodium lauryl sulfate is a water-soluble salt, produced as a white or cream powder, crystals, or flakes and is used as a wetting agent and detergent. Also known as dodecyl sodium sulfate, sodium lauryl sulfate is actually a mixture of sodium alkyl sulfates consisting chiefly of sodium lauryl sulfate. Sodium lauryl sulfate is also known as sulfuric acid monododecyl ester sodium salt. Furthermore, sodium lauryl sulfate is readily available from commercial sources such as Sigma or Aldrich in both solid form and as a solution. The solubility of sodium lauryl sulfate is about 1 gm per 10 ml/water. The fatty acids of coconut oil, consisting chiefly of lauric acid, are catalytically hydrogenated to form the corresponding alcohols. The alcohols are then esterified with sulfuric acid (sulfated) and the resulting mixture of alkyl bisulfates (alkyl sulfuric acids) is converted into sodium salts by reacting with alkali under controlled conditions of pH.

Alternative anionic surfactants include docusate salts such as the sodium salt thereof. Other suitable anionic surfactants include, without limitation, alkyl carboxylates, acyl lactylates, alkyl ether carboxylates, N-acyl sarcosinates, polyvalent alkyl carbonates, N-acyl glutamates, fatty acid, polypeptide condensates and sulfuric acid esters.

In other aspects of the disclosure amphoteric (amphipathic/amphiphilic surfactants), non-ionic surfactants and/or cationic surfactants are included in the co-processed compositions of the disclosure. Suitable pharmaceutically-acceptable non-ionic surfactants such as, for example, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters and simethicone.

Other suitable pharmaceutically-acceptable surfactants include acacia, benzalkonium chloride, cholesterol, emulsifying wax, glycerol monostearate, lanolin alcohols, lecithin, poloxamer, polyoxyethylene, and castor oil derivatives. The name and/or method of preparation of the surfactant utilized in the present disclosure is not determinative of the usefulness of the product. Other advantages are possible.

Highly polar molecules may also be utilized as the compressibility augmenting agent. Such highly polar molecules include certain dyes, particular those which may be capable of binding to the cellulose surface while thereafter creating a relatively hydrophobic environment due to the presence of a hydrophobic portion of the molecule (e.g., a hydrophobic tail) which “points away” from the cellulose surface and discourages hydrophilic surface-to-surface cellulose interactions, such as hydrogen-bonding. Preferably, the dye is one which is pharmaceutically acceptable for inclusion in solid dosage forms.

Examples of suitable dyes include Congo Red (chemical name: 3,3′-[[1,1′Biphenyl]-4,4′-diylbis-(azo)]bis[4-amino-1-naphthalenesulfouic acid] disodium salt; FD&C Red No. 40 (also known as “Allura Red”) (chemical name: Disodium salt of 6-hydroxy-5[(2-methyl-4-sulfophenyl)azo]-2-naphthalenesulfonic acid); FD&C Yellow No. 5 (common name: tartrazine) (chemical name: 5-oxo-1-(p-sulfophenyl)-4-[(p-sulfophenyl)azo]-2-pyrazoline-3-carboxylic acid, trisodium salt); FD&C Yellow No. 6 (common name: Sunset Yellow FCF) (chemical name: Disodium salt of 1-p-sulphophenylazo-2-naphthol-6-sulfonic acid); Ponceau 4R (chemical name: Trisodium-2-hydroxy-1-(4-sulfonato-1-naphthylazo) naphthalene-6,8-disulfonate); Brown HT (chemical name: Disodium 4,4′-(2,4-dihydroxy-5-hydroxymethyl-3,3-phenylene bisazo)di(napthalene-1-sulfonate)); Brilliant Black BN (Chemical name: Tetrasodium 4-acetamido-5-hydroxy-6-[7-sulfonato-4-(4-sulfonatophenylazo)-1-naphthylazo]naphthalene-1,7-disulfonate); Carmoisine (chemical name: Disodium 4-hydroxy-3-(4-sulfanato-1-naphythylazo) Naphthalene-1-sulfonate); Amaranth (chemical name: Trisodium 2-hydroxy-1-(4-sulfonato-1-naphthylazo) naphthalene-3,6-disulfonate); and mixtures thereof.

The usable concentration range for the selected surfactant depends in part upon not only its molecular weight but also its degree of foaming, particularly when present in agitated slurries which can be spray dried to form the desired particulate. Thus, in those aspects of the disclosure where surfactants other than sodium lauryl sulfate are co-processed with the NACET, it is to be understood that the surfactant can be present in an amount which enhances the compressibility of the NACET and yet does not have a degree of foaming which would substantially inhibit spray drying.

In an embodiment utilizing a spray-drying process, an aqueous dispersion of NACET and a compressibility augmenting agent (for example, a surfactant or silicon dioxide) is brought together with a sufficient volume of hot air to produce evaporation and drying of the liquid droplets. The highly dispersed slurry is pumpable and capable of being atomized. It is sprayed into a current of warm filtered air, which supplies the heat for evaporation and conveys a dried product to a collecting device. The air is then exhausted with the removed moisture. The resultant spray-dried powder particles may be approximately spherical in shape and may be relatively uniform in size, thereby possessing excellent flowability. The co-processed particles are not necessarily uniform or homogeneous. Other drying techniques such as flash drying, ring drying, micron drying, tray drying, vacuum drying, radio-frequency drying, and possibly microwave drying, may also be used.

Alternatively, all or part of the excipient may be subjected to a wet granulation with an active ingredient. A representative wet granulation includes loading the novel excipient particles into a suitable granulator, such as those available from Baker-Perkins, and granulating the particles together with the active ingredient, preferably using an aqueous granulating liquid. In some embodiments, a portion of the total amount of the novel excipient is wet granulated with the active ingredient, and thereafter the additional portion of the novel excipient is added to the granulate. In yet other embodiments, the additional portion of the novel excipient to be added to the excipient/active ingredient granulate may be substituted with other excipients commonly used by those skilled in the art, depending of course upon the requirements of the particular formulation.

In other embodiments of the disclosure, a further material is added to the NACET and/or compressibility augmenting agent. Such additional materials include silicon dioxides, non-silicon metal oxides, starches, starch derivatives, surfactants, polyalkylene oxides, cellulose A ethers, celluloses esters, mixtures thereof, and the like. Specific further materials which may be included in the aqueous slurry (and consequently in the resultant agglomerated microcrystalline cellulose excipient) are aluminum oxide, stearic acid, kaolin, polydimethylsiloxane, silica gel, titanium dioxide, diatomaceous earth, corn starch, high amylose corn starch, high amylopectin corn starch, sodium starch glycolate, hydroxylated starch, modified potato starch, mixtures thereof, and the like. These additives may be included in desired amounts.

In addition to one or more active ingredients, additional pharmaceutically acceptable excipients or other additives can be added prior to preparation of the final product. For example, if desired, any generally accepted soluble or insoluble inert pharmaceutical filler (diluent) material can be included in the final product (e.g., a solid dosage form). Such inert pharmaceutical filler may comprise a monosaccharide, a disaccharide, a polyhydric alcohol, inorganic phosphates, sulfates or carbonates, and/or mixtures thereof. Examples of suitable inert pharmaceutical fillers include sucrose, dextrose, lactose, xylitol, fructose, sorbitol, calcium phosphate, calcium sulfate, calcium carbonate, microcrystalline cellulose, mixtures thereof, and the like.

An effective amount of any generally accepted pharmaceutical lubricant may optionally be added at the time the medicament is added, or in any event prior to compression into a solid dosage form. In some embodiments, when a surfactant is included as part or all of the compressibility augmenting agent, an additional inclusion lubricant may not be necessary.

The complete mixture, in an amount sufficient to make a uniform batch of tablets, may then subjected to tableting in a conventional production scale tableting machine at normal compression pressures for that machine, e.g., about 1500-10,000 lbs./sq in. The mixture should not be compressed to such a degree that there is subsequent difficulty in its hydration when exposed to gastric fluid.

The average tablet size for round tablets is preferably about 50 mg to 500 mg and for capsule-shaped tablets about 200 mg to 2000 mg. However, other formulations prepared in accordance with the present disclosure may be suitably shaped for other uses or locations, such as other body cavities, e.g., periodontal pockets, surgical wounds, vaginally, rectally.

The solid dosage forms in the present disclosure include systemically active therapeutic agents, locally active therapeutic agents, disinfecting agents, chemical impregnants, cleansing agents, deodorants, fragrances, dyes, animal repellents, insect repellents, fertilizing agents, pesticides, herbicides, fungicides, and plant growth stimulants, and the like.

The tablets of the present disclosure may also contain effective amounts of coloring agents, (e.g., titanium dioxide, F.D. & C. and D. & C. dyes; see the Kirk-Othmer Encyclopedia of Chemical Technology, Vol. 5, pp. 857-884, which is hereby incorporated by reference in its entirety), stabilizers, binders, odor controlling agents, and preservatives.

Alternatively, the novel excipient can be utilized in other applications wherein it is not compressed. For example, the granulate can be admixed with an active ingredient and the mixture then filled into capsules. The granulate can further be molded into shapes other than those typically associated with tablets. For example, the granulate together with active ingredient can be molded to “fit” into a particular area in an environment of use (e.g., an implant). All such uses would be contemplated by those skilled in the art and are deemed to be encompassed within the scope of the appended claims.

For pharmaceutical formulations, pharmaceutically-acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the crosslinked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be in unit dosage form. In such form the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

When one or more compositions disclosed herein comprise a combination of a NACET and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from one or more compounds disclosed herein. Alternatively, those agents are part of a single dosage form, mixed together with the compounds disclosed herein in a single composition.

Examples of the binder include sucrose; white soft sugar; pregelatinized starch; partially pregelatinized starch; cellulose or a derivative thereof such as microcrystalline cellulose, methyl cellulose, ethyl cellulose, sodium carboxymethyl cellulose (carmellose sodium), hydroxyethyl cellulose, hydroxyethyl methyl cellulose, hydroxypropyl cellulose, low-substituted hydroxypropyl cellulose, hydroxypropyl methyl cellulose (hypromelloses such as hypromellose 2208, hypromellose 2906, and hypromellose 2910), other polysaccharides such as acacia, powdered acacia, agar, powdered agar, guar gum, tragacanth, powdered tragacanth, pullulan, and pectin; acrylic acid based polymer such as methacrylic acid copolymer L, methacrylic acid copolymer LD, methacrylic acid copolymer S, ethyl acrylate-methyl methacrylate copolymer dispersion, aminoalkyl methacrylate copolymer E, and aminoalkyl methacrylate copolymer RS; sodium alginate; purified gelatin; hydrolyzed gelatin powder; carboxyvinyl polymer; copolyvidone; povidone; polyvinyl alcohol. These binders may be used singly or in a combination of two or more. Among these, a cellulose derivative is preferable, and hydroxypropyl cellulose is more preferable. It should be noted that, when povidone is contained as a binder, the obtained tablet tends to have reduced photostability and storage stability. Therefore, it is more preferable if this component is substantially not contained.

The binder content is not particularly limited and is preferably about 0.1 to 20% by weight with respect to the weight of the tablet (when the tablet is coated, the weight of the uncoated tablet), and more preferably about 0.5 to 5% by weight.

The binder amount is not particularly limited and is preferably about 0.01 to 100 parts by weight per 1 part by weight of NACET, and more preferably about 0.1 to 50 parts by weight. By setting the content and amount of the binder as described above, the productivity and disintegration ability can be improved.

Examples of disintegrants include starch or a derivative thereof such as wheat starch, corn starch, potato starch, partially pregelatinized starch, sodium carboxymethyl starch, and hydroxypropyl starch; cellulose or a derivative thereof such as microcrystalline cellulose, carboxymethyl cellulose (carmellose), calcium carboxymethyl cellulose (carmellose calcium), croscarmellose sodium, and low-substituted hydroxypropyl cellulose; crospovidone; alginic acid; and bentonite. These disintegrants (c) may be used singly or in a combination of two or more. Among these, starch or a derivative thereof, and cellulose or a derivative thereof are preferable, and sodium carboxymethyl starch, carmellose calcium, croscarmellose sodium and low-substituted hydroxypropyl cellulose are more preferable. It should be noted that, when crospovidone is contained, the obtained tablet tends to have reduced photostability and storage stability. Therefore, it is more preferable if this component is substantially not contained.

Here, in the present specification, “low-substituted hydroxypropyl cellulose” is a derivative of cellulose including hydroxypropoxyl groups by about 5 to 16%. The amount of the hydroxypropoxyl groups in the low-substituted hydroxypropyl cellulose may be measured by a method listed in, for example, the Japanese Pharmacopeia. The low-substituted hydroxypropyl cellulose may be produced by a method known in the art, or a commercially available product thereof may also be used. Examples of commercially available products of the low-substituted hydroxypropyl cellulose include, but are not limited to, “LH series” and “NBD series” manufactured by Shin-Etsu Chemical Co., Ltd.

Furthermore, in the present specification, “hydroxypropyl cellulose” is a derivative of cellulose including hydroxypropoxyl groups by about 50 to 85%. The amount of the hydroxypropoxyl groups in the hydroxypropyl cellulose may be measured by a method listed in, for example, the Japanese Pharmacopeia. The hydroxypropyl cellulose may be produced by a method known in the art, or a commercially available product thereof may also be used. Examples of commercially available products of the hydroxypropyl cellulose include, but are not limited to, “HPC series” manufactured by Nippon Soda Co., Ltd.; and “Klucel series” manufactured by Hercules Inc.

In the present specification, “sodium carboxymethyl starch” is a derivative of starch including sodium about 6 to 11%.

The disintegrant content is not particularly limited and is preferably about 1 to 25% by weight with respect to the weight of the tablet (when the tablet is coated, the weight of the uncoated tablet), more preferably about 2 to 20% by weight, and still more preferably about 3 to 15% by weight.

Furthermore, the disintegrant amount is not particularly limited, and is preferably about 0.1 to 500 parts by weight per 1 part by weight of NACET, more preferably about 1 to 500 parts by weight, and still more preferably about 1 to 250 parts h weight. By setting the content and amount of the disintegrant as described above, the disintegration ability can be improved.

Examples of lubricants include stearic acid or a salt thereof such as stearic acid, aluminum stearate, calcium stearate, and magnesium stearate; carnauba wax; glycerol ester of fatty acid; hydrogenated oil; yellow beeswax; white beeswax; talc; sodium stearyl fumarate; and polyethylene glycol (macrogols such as macrogol 400, macrogol 600, macrogol 1500, macrogol 4000, and macrogol 6000). These lubricants may be used singly or in a combination of two or more. Among these, stearate, sucrose ester of fatty acid, and hydrogenated oil are preferable, and magnesium stearate is more preferable.

The lubricant content is not particularly limited and is preferably about 0.1 to 10% by weight with respect to the weight of the tablet (when the tablet is coated, the weight of the uncoated tablet), more preferably about 0.2 to 8% by weight, and still more preferably about 0.3 to 7% by weight.

The lubricant amount is not particularly limited and is preferably about 0.01 to 50 parts by weight per 1 part by weight of NACET, and more preferably about 0.02 to 30 parts by weight. By setting the content and amount of the lubricant (d) as described above, the tabletability can be improved.

The tablet of the present invention may comprise other components in addition to the excipient, the binder, the disintegrant, and the lubricant. Examples of other components include various additives applicable to tablets, such as colorants, pH adjusters, preservatives, absorbefacients, taste enhancers, antioxidants, buffers, chelating agents, abrasives, solvents, hardening agents, surfactants, sweeteners, fluidizers, brightening agents, and flavors. Those components may be used in an amount that does not adversely affect the present invention.

The tablet of the present invention may be used as an uncoated tablet that comprises the above-described components but does not have a coating layer provided thereon. A coated tablet (film-coated tablet) provided with a coating layer is preferable to achieve long-term storage stability and prevent degradation due to light or the like.

The coating layer may comprise pharmaceutical additives, such as a coating agent, plasticizer, dispersant, defoaming agent, and the like, usually used for coating (for providing a coat to) orally administrable pharmaceutical preparations.

Examples of additives include celluloses such as microcrystalline cellulose, methyl cellulose, ethyl cellulose, carmellose sodium, hydroxypropyl cellulose, and hydroxypropyl methyl cellulose (hypromellose) and derivatives thereof; polyethylene glycol (macrogol); polyvinyl alcohol; titanium oxide; and talc. These additives may be used singly or in a combination of two or more.

Among these, a combination of hydroxypropyl methyl cellulose (hypromellose), talc, and titanium oxide, which are components for coating agent, is preferable. It should be noted that, when polyethylene glycol (macrogol) exists in the coating layer, the obtained tablet tends to have reduced photostability and storage stability. Therefore, it is more preferable if polyethylene glycol (macrogol) is substantially not contained.

Furthermore, with regard to the coated tablet, by coloring the coating layer, photostability can be supplied to the coated tablet. Therefore, a colorant is preferably added to the coating agent for coating the tablet.

Examples of colorants include: iron oxides such as red ferric oxide, yellow ferric oxide, and black iron oxide; titanium oxide; beta-carotene; food blue No. 2; food blue No. 2 aluminum lake; and riboflavin.

The colorant may be suitably selected, or used in combination, depending on the color of the coated tablet prepared. For example, to obtain a white coated tablet, titanium oxide is used; to obtain a red coated tablet, a combination of titanium oxide and red ferric oxide is used; to obtain a yellow coated tablet, a combination of titanium oxide and yellow ferric oxide is used; to obtain a blue coated tablet, a combination of titanium oxide and food blue No. 2 aluminum lake is used; to obtain an orange coated tablet, a combination of titanium oxide, red ferric oxide, and yellow ferric oxide is used; to obtain a green coated tablet, a combination of titanium oxide, yellow ferric oxide, and black iron oxide, or a combination of titanium oxide, yellow ferric oxide and food blue No. 2 aluminum lake is used; and to obtain a purple coated tablet, a combination of titanium oxide, red ferric oxide and black iron oxide, or a combination of titanium oxide, red ferric oxide and food blue No. 2 aluminum lake is used. As described above, a coated tablet may be made into various colors.

The colorant content is preferably about 0.1 to 3% by weight with respect to the total weight of the coated tablet, and about 5 to 50% by weight with respect to the weight of the coating layer of the coated tablet.

The amount of a coating layer in tablet that is coated using the coating agent and a colorant that is contained if necessary is preferably about 1 to 10 parts by weight per 100 parts by weight of a tablet (uncoated tablet) before having a coating provided thereon.

Specific preferable examples of the additives contained in the tablet of the present invention include: as an excipient, at least one member selected from the group consisting of sugars, sugar alcohols, starches, and celluloses.

### Selected Examples of Formulations

1) NACET alone in a capsule—The dosage within each capsule can vary. Delivery of NACET in a capsule to a subject may be easier than injecting NACET directly into an animal, such as a mice or a human, or into cells.
2) NACET+Food (e.g., any food article)—NACET is pungent. The presence of any food article may aid in the ability of a subject to consume NACET.
3) NACET+excipients—NACET is hygroscopic and can be sticky. Therefore, it may be difficult to fill NACET into a capsule by itself. Adding excipients may aid in the manufacturing process to make the NACET-containing products for consumption. Nonlimiting examples to be added into a NACET formulation include, but are not limited to, excipients, flow agents, binding agents, lubricants, disintegrants, drying agents, and compressing agents.
4) NACET+diluent—The diluent can be anything used to dissolve the NACET in the formulation so that NACET can be used for injection, as a nasal spray or oral agent.
5) NACET+flavor/scent in a capsule, raw powder, tablet, or any other type of delivery method—NACET may have a strong sulfur smell and/or a horrid sulfur taste. Due to this pungent taste and smell, it may be helpful to add ingredients that can mask the smell and taste in a product.
6) NACET+Sweeteners—To mask the pungent taste of NACET, including a sweetener helps make the product viable. Unlimited examples of the sweeteners include, but are not limited to, Stevia, Sugar, Vanillin, etc.

In one embodiment, a formulation comprises NACET about 50 mg, glycine about 525 mg, selenium about 25 g, molybdenum about 50 g, one or more excipients selected from a flow agent, a lubricant, and a drying Agent such as MCC, silicon dioxide, or rice flour, etc., a flavor/scent agent to mask scents such as lemon or peppermint essential oils, etc., and a sweetener such as Stevia or Vanillin, etc. In another embodiment, a formulation comprises NACET about 125 mg, glycine about 600 mg, selenium about 25 g, molybdenum about 50 g, one or more excipients selected from a flow agent, a lubricant, and a drying Agent such as MCC, silicon dioxide, or rice flour, etc., a flavor/scent agent to mask scents such as lemon or peppermint essential oils, etc., and a sweetener such as Stevia or Vanillin, etc. In one embodiment, a formulation comprises NACET from about 50 mg to about 125 mg, and one or more excipients selected from a flow agent, a lubricant, and a drying Agent such as MCC, silicon dioxide, or rice flour, etc. In another embodiment, a formulation comprises NACET from about 50 mg to about 125 mg, and a flavor/scent agent to mask scents such as lemon or peppermint essential oils, etc. In one embodiment, a formulation comprises NACET from about 50 mg to about 125 mg, and a sweetener such as Stevia or Vanillin, etc. In another embodiment, a formulation comprises NACET from about 50 mg to about 125 mg, and a diluent. In one embodiment, a formulation comprises NACET from about 50 mg to about 125 mg, and NAC. In another embodiment, a formulation comprises NACET from about 50 mg to about 125 mg, and selenium about 25 g. In one embodiment, NACET from about 50 mg to about 125 mg, and a food item. In another embodiment, a formulation comprises NACET from about 50 mg to about 125 mg, one or more excipients selected from a flow agent, a lubricant, and a drying Agent such as MCC, silicon dioxide, or rice flour, etc., a flavor/scent agent to mask scents such as lemon or peppermint essential oils, etc., and a sweetener such as Stevia or Vanillin, etc. In one embodiment, a formulation comprises NACET from about 50 mg to about 125 mg, and glycine from about 525 mg to about 600 mg.

### EMBODIMENTS

Embodiment 1. A food composition comprising a food carrier and a prodrug of N-acetyl L-cysteine, wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 2. The food composition of embodiment 1, wherein said food composition is packaged as a beverage.

Embodiment 3. The food composition of embodiment 1, wherein said food composition is packaged as a solid food.

Embodiment 4. The food composition of embodiment 1, wherein said food composition is packaged as semi-solid food.

Embodiment 5. The food composition of embodiment 1, wherein said food composition is packaged as a food product selected from the group consisting of a snack bar, a cereal product, a bakery product, and a dairy product.

Embodiment 6. The food composition of embodiment 1, wherein said food carrier is milk.

Embodiment 7. The food composition of embodiment 1, wherein said food composition is a soft drink.

Embodiment 8. A food composition comprising:
(a) an effective amount of a prodrug of N-acetyl L-cysteine for modulating levels of serum glutathione or plasma glutathione or red blood cell glutathione or organ glutathione in a subject in need thereof; and
(b) a food carrier and/or one or more agents;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 9. The food composition of embodiment 8, wherein said food composition is packaged as a beverage.

Embodiment 10. The food composition of embodiment 8, wherein said food composition is packaged as a solid food.

Embodiment 11. The food composition of embodiment 8, wherein said food composition is packaged as semi-solid food.

Embodiment 12. The food composition of embodiment 8, wherein said food composition is packaged as a food product selected from the group consisting of a snack bar, a cereal product, a bakery product, and a dairy product.

Embodiment 13. The food composition of embodiment 8, wherein said prodrug is present in an amount effective to modulate said red blood cell glutathione levels.

Embodiment 14. The food composition of embodiment 8, wherein said prodrug is present in an amount effective to modulate said plasma or said serum glutathione levels.

Embodiment 15. The food composition of embodiment 8, wherein said prodrug is present in an amount effective to modulate said organ glutathione levels.

Embodiment 16. The food composition of any one of embodiments 1-15, wherein said prodrug is NACET.

Embodiment 17. A food supplement comprising a prodrug of N-acetyl L-cysteine and a food additive agent; wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 18. The food supplement of embodiment 17, wherein said prodrug is NACET.

Embodiment 19. A method of preparing a food supplement comprising a prodrug of N-acetyl L-cysteine and a food additive agent, comprising mixing said prodrug with said food additive agent, wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 20. The method of embodiment 19, wherein said food additive agent is selected from the group consisting of a sweetening agent, a flavoring agent, a coloring agent, a filling agent, a binding agent, a lubricating agent, and a preservative agent.

Embodiment 21. The method of embodiment 19 or embodiment 20, wherein said prodrug is NACET.

Embodiment 22. A foodstuff composition, comprising:
(a) a capsule comprising a prodrug of N-acetyl L-cysteine; and
(b) a food carrier;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 23. The foodstuff composition of embodiment 22, wherein said capsule comprises about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, 100 mg, about 200 mg, about 300 mg, about 400 mg, about 500 mg, about 600 mg, about 700 mg, about 800 mg, about 900 mg, or about 1,000 mg of said prodrug.

Embodiment 24. The food stuff composition of embodiment 22, wherein said capsule comprises from about 1 mg to about 1,000 mg of said prodrug.

Embodiment 25. The foodstuff composition of any one of embodiments 22-24, wherein said food carrier is a dough, a food bar, a cereal product, a bakery product, a beverage, a pasta, grains, an egg product, a snack, meat, fish, poultry, a fruit, or a vegetable, or combination thereof.

Embodiment 26. The foodstuff composition of embodiment 25, wherein said food far is a granola bar, a protein bar, or a candy bar; wherein said cereal product is s oatmeal cereal, breakfast cereal, or granola cereal, wherein said bakery product is bread, a cookie, a donut, a cracker, a bagel, a pastry, or a cake; wherein said beverage is a milk-based beverage, a sports drink, fruit juice, an alcoholic beverage, or bottled water; wherein said grains are rice, corn, oats, rye, wheat, or flour; and wherein said snack is a candy, chips, a gum, or a chocolate.

Embodiment 27. The food stuff composition of any one of embodiments 22-26, wherein said capsule further comprises one or more agents.

Embodiment 28. The foodstuff composition of embodiment 27, wherein said one or more agents comprise glycine, selenium, or molybdenum, or a combination thereof.

Embodiment 29. The foodstuff composition of embodiment 28, wherein said one or more agents comprise about 525 mg glycine.

Embodiment 30. The foodstuff composition of embodiment 28, wherein said one or more agents comprise about 525 mg glycine, about 25 μg selenium, and about 50 μg molybdenum.

Embodiment 31. The foodstuff composition of any one of embodiments 27-30, wherein said one or more agents comprises one or more essential oils.

Embodiment 32. The foodstuff composition of embodiment 31, wherein said one or more essential oils are anise oil, angelica oil, basil oil, bay oil, bay laurel oil, bergamot oil, bois de rose oil, California bay laurel oil, camphor oil, cananga oil, cannabis oil, cardamom oil, caraway oil, cedar oil, cedarwood oil, Chamaecyparis obtusa oil, chamomile oil, cinnamon oil, citronella oil, clove oil, copaiba balsam oil, cumin oil, clove oil, coriander oil, dill oil, eucalyptus oil, fennel oil, garlic oil, geranium oil, grapefruit oil, ginger oil, guaiac oil, hiba oil, Indonesian bay leaf oil, iris oil, Japanese mint oil, jasmine oil, lavender oil, lemon oil, lemongrass oil, linaloe oil, Lindera oil, marjoram oil, mandarin oil, melaleuca oil, Mexican bay leaf oil, mint oil, neroli oil, onion oil, orange oil, oregano oil, palmarosa sofia oil, patchouli oil, parsley oil, pepper oil, peppermint oil, perilla oil, Peru balsam oil, petitgrain oil, pine oil, pine needle oil, rose oil, rosemary oil, sage oil, sandalwood oil, spearmint oil, star anis oil, sweet orange oil, tangerine oil, tea seed oil, tea tree oil, thyme oil, tolu balsam oil, tuberose oil, turmeric oil, vetivert oil, West Indian bay tree oil, western mint oil, or wintergreen oil, or a combination thereof.

Embodiment 33. The foodstuff composition of any one of embodiments 22-32, wherein said prodrug is NACET.

Embodiment 34. A composition, comprising a solid form, a semi-solid form, a capsule, a gummy, or a liquid form, wherein each of said solid form, said semi-solid form, said capsule, said gummy, and said liquid form comprises:
(a) a prodrug of N-acetyl L-cysteine; and
(b) one or more agents;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 35. The composition of embodiment 34, wherein said prodrug is from about 10 to about 1000 mg.

Embodiment 36. The composition of embodiment 34, wherein said prodrug is about 50 mg.

Embodiment 37. The composition of any one of embodiments 34-36 or the food composition of any one of embodiments 8-16, wherein said one or more agents comprises glycine, selenium, or molybdenum, or a combination thereof.

Embodiment 38. The composition of embodiment 37, wherein said one or more agents comprise about 525 mg glycine.

Embodiment 39. The composition of embodiment 37, wherein said one or more agents comprise about 525 mg glycine, about 25 μg selenium, and about 50 μg molybdenum.

Embodiment 40. The composition of embodiment 37, wherein said one or more agents comprise from about 100 to about 900 mg glycine.

Embodiment 41. The composition of embodiment 37, wherein said one or more agents comprise from about 100 to about 900 mg glycine, from about 5 to about 50 μg selenium, and from about 10 to about 100 μg molybdenum.

Embodiment 42. The composition of any one of embodiments 34-41 or the food composition of any one of embodiments 8-15, wherein said one or more agents comprises peppermint, vanillin, silicon dioxide, or a vegetable capsule, or a combination there of.

Embodiment 43. The composition of any one of embodiments 34-41 or the food composition of any one of embodiments 8-16, wherein said one or more agents comprises one or more pharmaceutically acceptable excipients, a preservative, a flavoring agent, a vegetable capsule, a sweetener, a coloring agent, or one or more essential oils, or a combination thereof.

Embodiment 44. The composition of any one of embodiments 34-43, wherein said composition is a dough, a food bar, a cereal product, a bakery product, a beverage, a pasta, grains, an egg product, a snack, a soup, or combination thereof.

Embodiment 45. The composition of embodiment 44, wherein said food far is a granola bar, a protein bar, or a candy bar; wherein said cereal product is s oatmeal cereal, breakfast cereal, or granola cereal, wherein said bakery product is bread, a cookie, a donut, a cracker, a bagel, a pastry, or a cake; wherein said beverage is a milk-based beverage, a sports drink, fruit juice, an alcoholic beverage, or bottled water; wherein said grains are rice, corn, oats, rye, wheat, or flour; and wherein said snack is a candy, chips, a gum, a gummy candy, a jelly, or a chocolate.

Embodiment 46. The composition of embodiment 34, wherein said one or more agents comprises said one or more essential oils.

Embodiment 47. The composition of any one of embodiments 43-46, wherein said one or more essential oils are anise oil, angelica oil, basil oil, bay oil, bay laurel oil, bergamot oil, bois de rose oil, California bay laurel oil, camphor oil, cananga oil, cannabis oil, cardamom oil, caraway oil, cedar oil, cedarwood oil, Chamaecyparis obtusa oil, chamomile oil, cinnamon oil, citronella oil, clove oil, copaiba balsam oil, cumin oil, clove oil, coriander oil, dill oil, eucalyptus oil, fennel oil, garlic oil, geranium oil, grapefruit oil, ginger oil, guaiac oil, hiba oil, Indonesian bay leaf oil, iris oil, Japanese mint oil, jasmine oil, lavender oil, lemon oil, lemongrass oil, linaloe oil, Lindera oil, marjoram oil, mandarin oil, melaleuca oil, Mexican bay leaf oil, mint oil, neroli oil, onion oil, orange oil, oregano oil, palmarosa sofia oil, patchouli oil, parsley oil, pepper oil, peppermint oil, perilla oil, Peru balsam oil, petitgrain oil, pine oil, pine needle oil, rose oil, rosemary oil, sage oil, sandalwood oil, spearmint oil, star anis oil, sweet orange oil, tangerine oil, tea seed oil, tea tree oil, thyme oil, tolu balsam oil, tuberose oil, turmeric oil, vetivert oil, West Indian bay tree oil, western mint oil, or wintergreen oil, or a combination thereof.

Embodiment 48. The composition of any one of embodiments 34-47, wherein said composition comprises said capsule, and wherein said capsule comprises said prodrug and said one or more agents.

Embodiment 49. The composition of any one of embodiments 34-48, wherein said prodrug is NACET.

Embodiment 50. A composition, comprising:
(a) a prodrug of N-acetyl L-cysteine; and
(b) one or more agents;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof.

Embodiment 51. The composition of embodiment 50, wherein said prodrug is from about 10 to about 1000 mg.

Embodiment 52. The composition of embodiment 50, wherein said prodrug is about 50 mg.

Embodiment 53. The composition of any one of embodiments 50-52, wherein said one or more agents comprise glycine, selenium, or molybdenum, or a combination thereof.

Embodiment 54. The composition of embodiment 53, wherein said one or more agents comprise about 525 mg glycine.

Embodiment 55. The composition of embodiment 53, wherein said one or more agents comprise about 525 mg glycine, about 25 μg selenium, and about 50 μg molybdenum.

Embodiment 56. The composition of embodiment 53, wherein said one or more agents comprise from about 100 to about 900 mg glycine.

Embodiment 57. The composition of embodiment 53, wherein said one or more agents comprise from about 100 to about 900 mg glycine, from about 5 to about 50 μg selenium, and from about 10 to about 100 μg molybdenum.

Embodiment 58. The composition of any one of embodiments 50-57, wherein said one or more agents comprise a food carrier, one or more pharmaceutically acceptable excipients, a preservative, a flavoring agent, a vegetable capsule, a sweetener, a coloring agent, or one or more essential oils, or a combination thereof.

Embodiment 59. The composition of embodiment 58, wherein said food carrier is a dough, a food bar, a cereal product, a bakery product, a beverage, a pasta, grains, an egg product, a snack, a soup, meat, fish, poultry, a fruit, or a vegetable, or combination thereof.

Embodiment 60. The composition of embodiment 59, wherein said food far is a granola bar, a protein bar, or a candy bar; wherein said cereal product is s oatmeal cereal, breakfast cereal, or granola cereal, wherein said bakery product is bread, a cookie, a donut, a cracker, a bagel, a pastry, or a cake; wherein said beverage is a milk-based beverage, a sports drink, fruit juice, an alcoholic beverage, or bottled water; wherein said grains are rice, corn, oats, rye, wheat, or flour; and wherein said snack is a candy, chips, a gum, a gummy candy, a jelly, or a chocolate.

Embodiment 61. The composition of any one of embodiments 58-60, wherein said one or more essential oils are anise oil, angelica oil, basil oil, bay oil, bay laurel oil, bergamot oil, bois de rose oil, California bay laurel oil, camphor oil, cananga oil, cannabis oil, cardamom oil, caraway oil, cedar oil, cedarwood oil, Chamaecyparis obtusa oil, chamomile oil, cinnamon oil, citronella oil, clove oil, copaiba balsam oil, cumin oil, clove oil, coriander oil, dill oil, eucalyptus oil, fennel oil, garlic oil, geranium oil, grapefruit oil, ginger oil, guaiac oil, hiba oil, Indonesian bay leaf oil, iris oil, Japanese mint oil, jasmine oil, lavender oil, lemon oil, lemongrass oil, linaloe oil, Lindera oil, marjoram oil, mandarin oil, melaleuca oil, Mexican bay leaf oil, mint oil, neroli oil, onion oil, orange oil, oregano oil, palmarosa sofia oil, patchouli oil, parsley oil, pepper oil, peppermint oil, perilla oil, Peru balsam oil, petitgrain oil, pine oil, pine needle oil, rose oil, rosemary oil, sage oil, sandalwood oil, spearmint oil, star anis oil, sweet orange oil, tangerine oil, tea seed oil, tea tree oil, thyme oil, tolu balsam oil, tuberose oil, turmeric oil, vetivert oil, West Indian bay tree oil, western mint oil, or wintergreen oil, or a combination thereof.

Embodiment 62. The composition of any one of embodiments 58-60, wherein said one or more agents are said food carrier.

Embodiment 63. The composition of embodiment 61, wherein said one or more agents are said one or more essential oils.

Embodiment 64. The composition of any one of embodiments 58-61, wherein said one or more pharmaceutically acceptable excipients are a binder, a disintegrant, a lubricant, a filler, a diluent, a glidant, or a surface-active agent, or a combination thereof.

Embodiment 65. The composition of embodiment 64, wherein said one or more pharmaceutically acceptable excipients comprise microcrystalline cellulose (MCC).

Embodiment 66. The composition of embodiment 64 or embodiment 65, wherein said one or more pharmaceutically acceptable excipients further comprise magnesium stearate, and/or silicon dioxide.

Embodiment 67. The composition of any one of embodiments 50-66, wherein said one or more agents comprise vanillin.

Embodiment 68. The composition of any one of embodiments 50-67, wherein said one or more agents comprises a therapeutic agent.

Embodiment 69. The composition of any one of embodiments 50-68, wherein said prodrug is NACET.

### Bending NACET

NACET is sticky and has a melting point at about 44° C. The raw powder of NACET is stored at room temperature, and preferably avoids being placed under direct sunlight. In some embodiments, when blending with other ingredients, NACET is dispersed into excipients that can act as drying agents, such as, for example, silicon dioxide.

If the blender gets hotter than the melting point of NACET during the blending due to the length of blending, the operational temperature, or other measures, the NACET can melt and cause the entire blend to become wet and sticky, thus necessitating the addition of more drying agents, such as, for example, silicon dioxide, rice flour, microcrystalline cellulose (MCC) or other excipients.

In some embodiments, formulations not allowing the ingredient of scent maskers, such as, for example, essential oils or other maskers, to dissipate (such as capsules), are preferred. Other blending includes, but is not limited to, making the NACET into tablets, food products, raw powders, etc.

In further embodiments of the disclosure, more than one compressibility augmenting agent is used. Thus, for example, two or more compressibility enhancing agents are used which provide an effect by different mechanisms.

### Materials and Chemicals

N-Acetyl-L-cysteine (NAC), sodium nitrite (NaNO₂), anhydrous ethanol and other anhydrous reagents, and supplies/materials are obtained from commercial sources such as Sigma Aldrich, Fisher Scientific, VWR International, DOW Corporation, etc.

### EXAMPLES

### Example 1: Preparation of NACET

A suspension of N-acetyl-L-cysteine (3.26 g) in dry ethanol (12 mL) under nitrogen is stirred for 15 minutes and treated dropwise with concentrated sulfuric acid (0.08 mL) at room temperature with vigorous stirring. After 22 hours of stirring, the mixture is treated with water (2.5 mL) and the volatiles are removed under reduced pressure. The resulting residue is diluted with ethyl acetate (20 mL), washed with aqueous saturated sodium bicarbonate (150 mL) and the layers are allowed to separate.

The organic layer is separated from the aqueous layer and dried over anhydrous sodium sulfate. The aqueous layer is re-extracted with ethyl acetate (2×10 mL). The combined organic extract is filtered and concentrated in vacuo to yield N-Acetyl-L-cysteine ethyl ester as a solid.

Similarly, other esters of NAC, for example, methyl ester, n-propyl ester, isopropyl ester, and n-butyl ester may be prepared similarly to the procedure described for NACET by replace the ethanol with methanol, n-propanol, isopropanol, and n-butanol, respectively and by varying the procedures according to the alcohols used in each experiment.

### Example 2: Preparation of SNACET

NACET (0.5 g) prepared in Example 1 is dissolved in 10 mL of deionized water, followed by the addition of 2M hydrochloric acid (1.0 M). The reaction mixture is cooled in an ice bath for about 30 minutes. Then NaNO₂ (0.2 g) is added. The resulting mixture is stirred for 10 minutes. Precipitates that formed are filtered, washed with ice-cold water, dried in a vacuum desiccator to provide S-nitroso-N-acetylcysteine ethyl ester (SNACET) as a solid. The solid is stored at −80° C. in the dark, optionally placed in a sealed container filled with inert gas such as argon or nitrogen.

### Example 3: Measurement of Concentrations of Free Sulfhydryl Groups in Blood Plasma Proteins

The concentration of free sulfhydryl groups in plasma proteins are assessed according to the methods reported in (1) K. Karolczak, et al., Homocysteine is a novel risk factor for suboptimal response of blood platelets to acetylsalicylic acid in coronary artery disease: a randomized multicenter study. Phamacol Res. 203; 74:7-22; and (2) in Y. Ando, et al., Sulfhydryl and disulfide groups of platelet membranes. I. Determination of sulfhydryl groups. Biochim Biophys Acta. 1973; 311:26-37.

### Example 4: Red Blood Cell Glutathione and Glutathione Peroxidase Assays

Commercial GSH assay kit (Oxford Biomedical Research Inc.) is used for evaluation of Red blood cell glutathione level and glutathione peroxidase activity. Manufacturer's instructions are strictly followed during the assay. Five hundred μL of whole blood is mixed with 500 μL of deionized water and hemolysates, and the GSH/GSSG ratio is calculated. Hemolysates of previous step are used to measure GPX of erythrocytes. Complied with the Cayman's Glutathione Peroxidase Assay Kit guide and achieved GPX activity.

### Example 5: Other Assays

Additional assays are conducted. BIOXYTECH GSH-400 and BIOXYTECH H2O2-560 Assay kit (OXIS International, Inc., Portland, U.S.A.) are used to measure concentrations of reduced glutathione and hydrogen peroxide. Glutathione Assay Kit (Cayman Chemical Company, U.S.A.) is used to determine serum total glutathione level.

### Example 6: Uncoated Tablet

NACET, lactose, corn starch, microcrystalline cellulose, and hydroxypropylcellulose are weighed and mixed. A separately prepared aqueous solution of hydroxypropylcellulose is added to the powder mixture, followed by wet kneading granulation. After drying and sizing the result, magnesium stearate is added thereto and mixed. The resulting mixture is compressed using a single tableting machine equipped with a punch of diameter 7.0 mm in such a manner that the weight of the tablet becomes 250 mg, obtaining uncoated tablets containing 50 mg of NACET per tablet.

### Example 7: Pharmacokinetic Profile of NAC, NACET, and Product X in Healthy Men and Women

### Experimental Design

A double-blind, single ingestion, crossover study of NAC, NACET and Product X.

The present study compares three different formulations containing NAC or NACET, with regards to acute absorption over a 24-hour period, following a single ingestion. The tree formulations are (1) traditional NAC only, (2) NACET only, and (3) a product containing a combination of NACET, glycine, and two minerals with antioxidant potential (selenium and molybdenum) that might enhance the effect of NACET. Subjects report to the lab on three different occasions to consume the products, using a randomized cross-over design, and blood is collected periodically (including at 8 hours post-dosing and then again at 24 hours post-dosing) per standard, routinely used pharmacokinetic (PK)/pharmacodynamic (PD) study protocols for evaluation of circulating glutathione concentrations.

### Treatment

Single dose ingestion of the following drug choices, separated by approximately one week between each drug treatment. Each drug treatment is delivered in a serving of 3 capsules as follows:

NAC Dose: 375 mg (125 mg per capsule).

NACET Dose: 375 mg (125 mg per capsule).

Product X (including NACET, glycine, selenium, molybdenum) Dose: 375 mg NACET, 1800 mg glycine, 75 μg selenium, 150 μg molybdenum (125 mg NACET, 600 mg glycine, 25 μg selenium, 50 g molybdenum per capsule).

### Subjects

Subjects (N=8) report to the lab following an overnight fast (10 hours) in the morning hours. Testing begins between 6:00 am and 8:00 am. Upon arrival, subjects are asked to void and female subjects will be escorted to a private restroom within the lab where they self-collect a urine sample to perform a pregnancy test. A female investigator privately reviews the results with the subject. Immediately following review, the test strip is discarded in a private trash can. If a pregnancy test is positive, the subject is advised to make an appointment to see their personal physician (OBGYN). After urine collection, the subject rests quietly for 10 minutes before their vital signs (heart rate, blood pressure) are taken. Subjects review their medication/dietary supplement use with investigators and indicate any changes since their previous visit. A blood sample is collected as described below.

Subjects receive their assigned treatment for the day, as indicated above (NAC, NACET, or NACET plus), and heart rate, blood pressure, and blood samples (for measurement of glutathione concentrations, as well as lipid and protein oxidation) are obtained at baseline (pre-dose), and 0.25, 0.5, 1, 1.5, 2, 4, 6, 8, and 24 h post-dose (total of 10 samples per dose trial).

Standardized meals (meal replacement food bars [e.g., Clif Builder] or shakes [e.g., Orgain]) are provided to subjects after sample collection at hour 2 and hour 6. Subjects are also provided with meal replacement bars and shakes to consume following the 8 hour sample collection (during their time outside of the lab, prior to the 24 hour visit). No other food other than what is provided to subjects is allowed during each study day, including both time spent in the lab and time spent outside of the lab. Water is allowed ad libitum. Subjects return the following morning for the 24 hour blood collection (e.g., via single venipuncture), again in a 10 hour fasted state.

### Participants and Inclusion Criteria

Subjects are healthy, recreationally active males (n=4) and females (n=4), 18-45 years old, body mass index (BMI) 18-29.9 kg/m² (non-obese subjects only), with normal vital signs (heart rate, systolic and diastolic blood pressure), no history of use of illicit drugs or other substances of abuse within 12 months of the screening visit, no tobacco use for >90 days prior to screening, and no history of cardiovascular disease; <400 mg caffeine intake per day.

In some embodiments, subjects are:
Age 18-45 years
   Individuals under the age of 18 are excluded because they are legally minors.
   To maintain a homogenous subject sample, the age range is capped at 45 years of age since multiple metabolic changes become more prevalent in the fifth and sixth decade of life, including issues with absorption of nutrients. This strategy permits more meaningful biological comparisons.
Male or female (4 of each sex)
Recreationally active (2 or more days per week of exercise for >30 minutes/day)
Body mass index (BMI) between 18-29.9 kg/m² (not obese)
   Obese individuals are excluded to reduce the variability in nutrient absorption. A more homogenous sample can better detect significant differences in experimental results.
Non-tobacco user
   Tobacco users are excluded in order to maintain more homogeneity in our sample of otherwise healthy subjects.
No known chronic disease, including diagnosed digestive disorders.
   These may impact nutrient absorption.
No known sensitivity or allergy to any of the study products.
No consumption of alcohol-containing beverages within 24 hours of testing.
No consumption of caffeine-containing beverages within 24 hours of testing.
Without active infection or illness of any kind.
If female, not lactating, pregnant or planning to become pregnant during the study
   Females take a urine pregnancy test on the morning of each test day.
Able to fast overnight (greater than 10 hours).
   Required for study
Willing to adhere to all study procedures, as indicated in the consent form

### Objectives

To characterize whole blood glutathione (GSH and GSSG) pharmacokinetics in human subjects
To determine oxidative stress biomarkers before and following acute ingestion of test products—as an increase in glutathione may impact oxidative stress markers
To measure the subjective mood and hemodynamic responses of human subjects following a single ingestion of test products. This is done as a precautionary “safety” measure since these measures may not be altered with treatment.

Primary Outcome Variables (related to blood glutathione)
Area under the concentration curve from time 0 to infinity (AUC0-inf)
Terminal Half-life (t_{1/2})
Peak concentration (Cmax)
Time to maximum concentration (Tmax)
Lag time (Tlag)
Apparent volume distribution during terminal elimination phase (Vz/F)
Oral Clearance (CL/F)

Secondary Outcome Variables (obtained at baseline, hours 2, 4, 8, and 24)
Blood Malondialdehyde
Blood Advanced Oxidation Protein Products
Brunel Mood Scale (see Table 1)
Subjective feelings of energy, mood, motivation, focus, attention, irritability, jitters (see Questionnaire 1)

Tertiary Outcome Variables (obtained at all noted blood collection times)
Heart rate
Systolic blood pressure
Diastolic blood pressure

During the second and third testing visit, the subject will undergo the same procedures, except they will receive one of the treatments they have yet to receive.

### Blood Collection

Blood is collected using a sterile venous indwelling catheter placed into a forearm vein and remaining patent with the use of saline solution as needed (through hour 8 only). The catheter is removed at hour 8 and the subjects return the following morning for the 24 h sample to be taken via standard venipuncture. Again, for the 24-hour time point, a single venipuncture is used for blood collection.

The above procedures are used to collect blood samples from subjects at each of the 3 test visits. Therefore, a total of 10 blood samples will be collected from each subject each dose trial, and 30 samples over the three visits. Approximately 10 mL of blood (2 teaspoons) is taken from subjects at each collection time. The collected samples are processed and plasma/serum stored in multiple aliquots at −70° C. until they are analyzed for glutathione, protein and lipid peroxidation products.

Safety assessments include monitoring of adverse events (AEs) and vital signs. For each AE recorded, an intensity level (i.e., mild, moderate, or severe) is assigned. Mild AEs are defined as “generally did not interfere with daily activities, required no special treatment, and were transient in nature.” Moderate AEs are defined as “impaired daily activities but were easily resolved.” Severe AEs are defined as “interrupted daily activities and required vigorous therapeutic intervention.”

### Physical Activity and Diet

Subjects follow their usual activity patterns over the course of the study period but refrain from strenuous activity, alcohol, and caffeine for the 24 hours preceding each lab test day. Dietary intake remains similar over the entire study period. Standardized meals (meal replacement food bars or shakes) are provided to subjects after sample collection at hour 2 and hour 6. Subjects are also provided with adequate meal replacement bars (2-3) and shakes (2-3) to consume following the 8-hour sample collection (during their time outside of the lab). No food other than what is provided to subjects is allowed during each study day, including both time spent in the lab and time spent outside of the lab. The beverage that the subjects consume is water. Subjects return the following morning for the 24 hour blood collection, again in a 10 hour fasted state. The same volume of meal replacement bars or shakes are consumed by each subject during each visit (both in lab and outside of lab). They also consume standard prepackaged meals during the day prior to each 8.5 hour test day (these are meal replacement drinks, bars, and fruit and mixed nuts).

### Descriptive Variables

Age
Height
Body mass
BMI
Resting heart rate
Resting blood pressure

Product provided by Sponsor (additional quantities included to account for attrition) Fifteen servings of each of the three test products (NAC, NACET and Product X)

### Data Analysis

The data are presented as mean±standard deviation (SD). Analysis includes a repeated measures analysis of variance (ANOVA) with post hoc testing as appropriate. Differences between men and women may also be investigated. The area under the curve is calculated and the standard procedures for computing pharmacokinetic data are followed for modeling, as done in He et al., J Caffeine Res. 2017 Sep. 1; 7(3):95-102 or Schilling et al., 2013 BMC Pharmacol Toxicol. 2013 Oct. 4; 14:52.

Glutathione analysis, total thiols levels, malondialdehyde (MDA) analysis, advanced oxidation protein products (AOPP) analysis, protein carbonyls (PC) concentrations, and/or glutathione peroxidase (GSH-Px) activity of all collected sample were performed. Entry of all obtained/determined data in a database.

Statistical analyses are performed on the data collected.

### (1) GSH Levels

To compare the effects of different treatments on GSH levels over time, an ANOVA is conducted. The treatment group is treated as a categorical independent variable, and time is treated as a within-subjects factor. Post-hoc tests with appropriate adjustments (e.g., Bonferroni correction) are performed to identify significant differences between specific treatment groups and time points.

### (2) Acute Absorption

To compare the effects of different treatments on acute absorption levels at each time point separately, a one-way ANOVA is conducted. The treatment group is treated as a categorical independent variable. Post-hoc tests with appropriate adjustments (e.g., Scheffe's method) are performed to identify significant differences between treatment groups.

### The Brunel Mood Scale Questionnaire

Below is a list of words that describe feelings people have. Please read each one carefully and then circle the answer that best describes HOW YOU FEEL RIGHT NOW. Make sure you respond to every word.

TABLE 1
Brunel Mood Scale Questionnaires:

| | | | | | Scoring for the BRUMS-32 (add the responses | | |
|---|---|---|---|---|---|---|---|
| Not | A | | Quite | | for the responses to each of the subscales) | | |
| at all | little | Moderately | a bit | Extremely | Subscale | Scores | Total |

| | | | | | | | |
|---|---|---|---|---|---|---|---|
| 1. Active | 0 | 1 | 2 | 3 | 4 | | |
| 2. Alert | 0 | 1 | 2 | 3 | 4 | Anger | ANGRY (3)_{——} |
| 3. Angry | 0 | 1 | 2 | 3 | 4 | | +ANNOYED (4)_{——} |
| 4. Annoyed | 0 | 1 | 2 | 3 | 4 | | +BAD TEMPERED (6)_{——} |
| 5. Anxious | 0 | 1 | 2 | 3 | 4 | | +BITTER (7)_{——} |
| 6. Bad tempered | 0 | 1 | 2 | 3 | 4 | Tension | ANXIOUS (5)_{——} |
| 7. Bitter | 0 | 1 | 2 | 3 | 4 | | +NERVOUS (20)_{——} |
| 8. Calm | 0 | 1 | 2 | 3 | 4 | | +PANICKY (21)_{——} |
| 9. Cheerful | 0 | 1 | 2 | 3 | 4 | | +WORRIED (30)_{——} |
| 10. Composed | 0 | 1 | 2 | 3 | 4 | Depression | DEPRESSION (13)_{——} |
| 11. Confused | 0 | 1 | 2 | 3 | 4 | | +DOWNHEARTED (14)_{——} |
| 12. Contented | 0 | 1 | 2 | 3 | 4 | | +MISERABLE (19)_{——} |
| 13. Depressed | 0 | 1 | 2 | 3 | 4 | | +UNHAPPY (28)_{——} |
| 14. Downhearted | 0 | 1 | 2 | 3 | 4 | Vigour | ACTIVE (1)_{——} |
| 15. Energetic | 0 | 1 | 2 | 3 | 4 | | +ALERT (2)_{——} |
| 16. Exhausted | 0 | 1 | 2 | 3 | 4 | | +ENERGETIC (15)_{——} |
| 17. Happy | 0 | 1 | 2 | 3 | 4 | | +LIVELY(18)_{——} |
| 18. Lively | 0 | 1 | 2 | 3 | 4 | Fatigue | EXHAUSTED(16)_{——} |
| 19. Miserable | 0 | 1 | 2 | 3 | 4 | | +SLEEPY (25)_{——} |
| 20. Nervous | 0 | 1 | 2 | 3 | 4 | | +TIRED (26)_{——} |
| 21. Panicky | 0 | 1 | 2 | 3 | 4 | | +WORN-OUT (29)_{——} |
| 22. Relaxed | 0 | 1 | 2 | 3 | 4 | Confusion | CONFUSED (11)_{——} |
| 23. Restful | 0 | 1 | 2 | 3 | 4 | | +UNCERTAIN (27)_{——} |
| 24. Satisfied | 0 | 1 | 2 | 3 | 4 | | +MIXED-UP (31)_{——} |
| 25. Sleepy | 0 | 1 | 2 | 3 | 4 | | +MUDDLED (32)_{——} |
| 26. Tired | 0 | 1 | 2 | 3 | 4 | Happy | CHEERFUL (9)_{——} |
| 27. Uncertain | 0 | 1 | 2 | 3 | 4 | | +CONTENT (12)_{——} |
| 28. Unhappy | 0 | 1 | 2 | 3 | 4 | | +HAPPY (17)_{——} |
| 29. Worn-out | 0 | 1 | 2 | 3 | 4 | | +SATISFIED (24)_{——} |
| 30. Worried | 0 | 1 | 2 | 3 | 4 | Calmness | CALM (8)_{——} |
| 31. Mixed-up | 0 | 1 | 2 | 3 | 4 | | +COMPOSED (10)_{——} |
| 32. Muddled | 0 | 1 | 2 | 3 | 4 | | +RELAXED (22)_{——} |
| | | | | | | | +RESTFUL (23)_{——} |

### Another Questionnaire

Directions: This questionnaire asks about patient's current feelings of the indicated items shown in the questionnaire below. The goal is to determine how each patient feels at the time point, even if it is different than how each patient usually feels. Therefore, it is important that the patient focuses on how him/herself feels at the time point. Place a vertical line on the horizontal line close to the number that best describes the overall feelings for each category of the questionnaire shown below.

### Questionnaire:

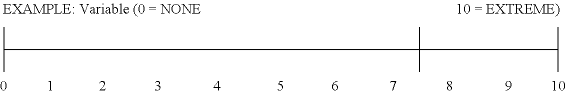

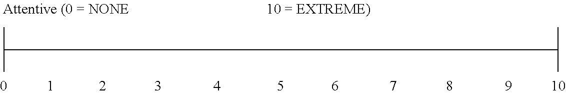

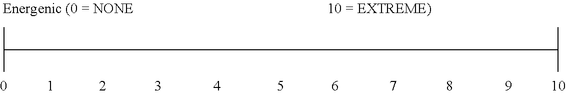

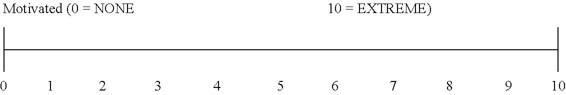

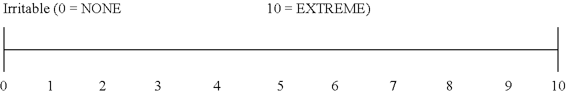

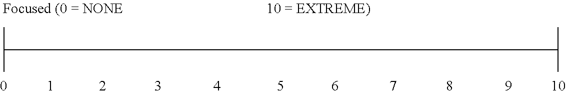

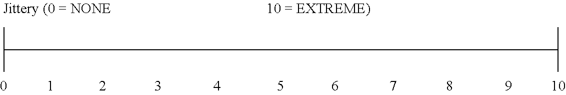

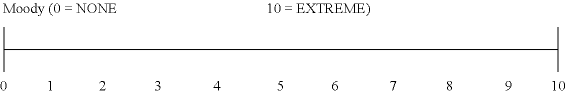

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-30. (canceled)

31. A food composition comprising:
(a) a prodrug of N-acetyl L-cysteine for modulating levels of serum glutathione or plasma glutathione or red blood cell glutathione or organ glutathione in a subject in need thereof; and
(b) a food carrier and/or one or more agents;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), S-nitroso-N-acetyl L-cysteine isopropyl ester (SNACIPE), or a combination thereof, and
wherein said prodrug is present in an amount effective to modulate said red blood cell glutathione levels, modulate said plasma or said serum glutathione levels, or modulate said organ glutathione levels.

32. The food composition of claim 31, wherein said food composition is packaged as a beverage, a solid food, or a semi-solid food.

33. The food composition of claim 31, wherein said prodrug is NACET.

34. The food composition of claim 31, wherein said one or more agents comprise glycine, selenium, molybdenum, or any combination thereof.

35. A foodstuff composition, comprising:
(a) a prodrug of N-acetyl L-cysteine in a suitable form;
(b) one or more agents; and
(c) a food carrier;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), S-nitroso-N-acetyl L-cysteine isopropyl ester (SNACIPE), or a combination thereof; and
wherein said one or more agents comprise glycine, selenium, molybdenum, or any combination thereof.

36. The foodstuff composition of claim 35, wherein said foodstuff composition comprises about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, 100 mg, about 200 mg, about 300 mg, about 400 mg, about 500 mg, about 600 mg, about 700 mg, about 800 mg, about 900 mg, or about 1,000 mg of said prodrug.

37. The foodstuff composition of claim 35, wherein said food carrier is a dough, a food bar, a cereal product, a bakery product, a beverage, a pasta, grains, an egg product, a snack, meat, fish, poultry, a fruit, or a vegetable, or combination thereof.

38. The food stuff composition of claim 35, wherein said suitable form is a liquid form, a gel form, a semi-liquid form, a semi-solid form, or a solid form.

39. The foodstuff composition of claim 38, wherein said suitable form comprises a tablet form, a capsule form, an oral gel form, a food form, a chewable form, a non-chewable form, a slow- or sustained-release form, or a non-slow- or non-sustained-release from.

40. The foodstuff composition of claim 35, wherein said prodrug is NACET.

41. The foodstuff composition of claim 35, wherein said prodrug is present in an amount effective to modulate a glutathione level, wherein said glutathione level comprises a red blood cell glutathione level, a plasma glutathione level, a serum glutathione level, an organ glutathione level, or any combination thereof.

42. A composition, comprising: (a) a prodrug of N-acetyl L-cysteine; and (b) one or more agents;
wherein said prodrug is N-acetyl L-cysteine ethyl ester (NACET), N-acetyl L-cysteine methyl ester (NACME), N-acetyl L-cysteine butyl ester (NACBE), N-acetyl L-cysteine n-propyl ester (NACPE), N-acetyl L-cysteine isopropyl ester (NACIPE), S-nitroso-N-acetyl L-cysteine ethyl ester (SNACET), S-nitroso-N-acetyl L-cysteine methyl ester (SNACME), S-nitroso-N-acetyl L-cysteine butyl ester (SNACBE), S-nitroso-N-acetyl L-cysteine n-propyl ester (SNACPE), or S-nitroso-N-acetyl L-cysteine isopropyl ester (SNACIPE), or a combination thereof, and
wherein said one or more agents comprise glycine, selenium, or molybdenum, or a combination thereof.

43. The composition of claim 42, wherein said prodrug is from about 10 to about 1000 mg.

44. The composition of claim 42, wherein said prodrug is NACET.

45. The composition of claim 42, wherein said one or more agents comprise from about 100 to about 900 mg glycine, from about 5 to about 50 μg selenium, or from about 10 to about 100 μg molybdenum, or a combination thereof.

46. The composition of claim 42, comprising from about 10 to about 1000 mg NACET, from about 100 to about 900 mg glycine, from about 5 to about 50 μg selenium, and from about 10 to about 100 μg molybdenum.

47. The composition of claim 42, wherein said one or more agents further comprise one or more pharmaceutically acceptable excipients, wherein said one or more pharmaceutically acceptable excipients are a binder, a disintegrant, a lubricant, a filler, a diluent, a glidant, or a surface-active agent, or a combination thereof, and wherein said one or more pharmaceutically acceptable excipients comprise microcrystalline cellulose (MCC).

48. The composition of claim 47, wherein said one or more pharmaceutically acceptable excipients further comprise magnesium stearate, and/or silicon dioxide.

49. The composition of claim 42, wherein said composition is a solid form, a semi-solid form, a liquid form, a gel form, a semi-liquid form, a capsule, a tablet, a pill, a lozenge, a gummy, or a liquid form.

50. The composition of claim 42, wherein said prodrug is present in an amount effective to modulate a glutathione level, wherein said glutathione level comprises a red blood cell glutathione level, a plasma glutathione level, a serum glutathione level, an organ glutathione level, or any combination thereof.
